(19) United States
(12) Patent Application Publication
(10) Pub. No.: US 2016/0158189 A1
(43) Pub. Date: Jun. 9, 2016

(54) SENSITIZATION OF CANCER CELLS TO APOPTOSIS INDUCTION BY FLAVAGLINES AND 5-HYDROXY-FLAVONES

(71) Applicant: DEUTSCHES KREBSFORSCHUNGSZENTRUM, Heidelberg (DE)

(72) Inventors: Min LI-WEBER, Bad Duerkheim (DE); Peter KRAMMER, Heidelberg (DE)

(21) Appl. No.: 14/905,442

(22) PCT Filed: Jul. 15, 2014
(86) PCT No.: PCT/EP2014/065095

§ 371 Date: Jan. 15, 2016

(30) Foreign Application Priority Data
Jul. 17, 2013 (EP) 13176877.2

(51) Int. Cl. A61K 31/343; A61K 31/352

(52) U.S. Cl. CPC A61K 31/343; A61K 31/352

(57) ABSTRACT

The present invention relates to a combined preparation for simultaneous, separate or sequential use comprising at least one flavagline or a pharmaceutically acceptable salt thereof; and/or at least one 5-hydroxy-flavone or a pharmaceutically acceptable salt thereof; and at least one agent activating the intrinsic pathway of apoptosis for use in the treatment of cancer. The N present invention further relates to a medicament comprising the combined preparation of the present invention, as well as to a kit comprising the combined preparation or a medicament according to the present invention and a means for administering at least one of its components. Moreover, the present invention relates to a method of inhibiting a cancer cell, comprising contacting said cancer cell with the combined preparation of the present invention, and thereby inhibiting said cancer cell.

The present invention relates to a combined preparation for simultaneous, separate or sequential use comprising at least one flavagline or a pharmaceutically acceptable salt thereof; and/or at least one 5-hydroxy-flavone or a pharmaceutically acceptable salt thereof; and at least one agent activating the intrinsic pathway of apoptosis for use in the treatment of cancer. The present invention further relates to a medicament comprising the combined preparation of the present invention, as well as to a kit comprising the combined preparation or a medicament according to the present invention and a means for administering at least one of its components. Moreover, the present invention relates to a method of inhibiting a cancer cell, comprising contacting said cancer cell with the combined preparation of the present invention, and thereby inhibiting said cancer cell.

Cancer constitutes the fourth leading cause of death in Western countries. As the average age in the Western population steadily rises, so do cancer-related deaths indicating that cancer will be one of the most common causes of death in the 21st century. The aggressive cancer cell phenotype is the result of a variety of genetic and epigenetic alterations leading to deregulation of intracellular signaling pathways. Cancer cells commonly fail to undergo so-called “programmed cell death” or “apoptosis”, a signaling process that plays a key role in preventing cell tissues from abnormal growth.

Hematological malignancies are cancers that primarily affect cells in blood, bone marrow, spleen and lymph nodes. They are caused by abnormal proliferation of cells of the immune system or their precursor cells. There are two subtypes of hematological malignancies, leukemia and lymphoma.

Leukemia is characterized by an overproduction of blood cells, usually leukocytes. Lymphoblastic leukemia is caused by the abnormal proliferation of lymphocytes. The major types of lymphocytes are the T-lymphocytes, B-lymphocytes and natural killer cells. Myeloid leukemia is caused by abnormal proliferation of bone marrow derived myeloid cells. Both types of leukemia can be separated into chronic and acute diseases. Acute forms of leukemia are characterized by the rapid build up of relatively immature cell types. They usually progress rapidly and kill the patient within a few weeks or months after diagnosis if left untreated. Acute lymphoblastic leukemia (ALL) is the most common type of childhood cancer. Chronic forms of leukemia are caused by relatively well differentiated cells. They often progress only slowly over years. In many cases it is sufficient to monitor the progress of the disease and to initiate treatment only when the symptoms start to impair the patient's quality of life.

A special type of leukemia is human T-cell leukemia virus type I (HTLV-1)-associated adult T-cell leukemia/lymphoma (ATL). This is a malignancy of the clonal proliferation of infected mature CD4+ T-cells. Primary HTLV-1-ATL samples and ATL cell lines derived from HTLV-1-infected patients are more resistant to TRAIL-and CD95L-mediated apoptosis as compared to non-HTLV-infected leukemic cells (Hasegawa H et al., 2005, British Journal of Haematology, 128: 253-265; Krueger et al., 2006, Blood 107: 3933-3939; Matsuda et al., 2005, Journal of Virology 79: 1367-1378). Worldwide HTLV-1 has infected 15-20 million people. Patients have a poor prognosis after disease development with a survival range of less than one year (Matsuoka and Jeang, 2007, Nature Reviews Cancer 7: 270-280).

Lymphoma are cancers of the lymphatic system, causing tumors in lymph nodes, the spleen, or in other parts of the lymphatic system. The main types of lymphoma are Hodgkin lymphoma and Non-Hodgkin lymphoma. Hodgkin lymphoma is characterized histopathologically by the presence of multinucleated Reed-Sternberg cells in tumor preparations. Non-Hodgkin lymphoma is the generic term used for all lymphoma which are not Hodgkin lymphoma, including diffuse large B-cell lymphoma, follicular lymphoma, marginal zone lymphoma, peripheral T-cell lymphoma, small cell B-cell lymphoma, and mantle cell lymphoma.

Three modes of cancer therapy are available. Curative surgery attempts to remove the tumor completely. This is only possible as long as there are no metastases. Sometimes surgery may be an option for the treatment of metastases if there are only few and they are easily accessible. Radiotherapy uses ionizing radiation, typically y-radiation, to destroy the tumor. Radiation therapy is based on the principle that tumor cells with their high metabolic rates are especially susceptible to radiation induced cell damage. The anti-tumor effect of radiation therapy has to be weighted against the damage to the surrounding healthy tissue. Thus, possible tissue damage can rule out this option in some cases due to the damage to healthy tissues to be feared. Furthermore, radiation therapy is limited to cases where the primary tumor has not yet spread or where only few metastases are present. Radiation therapy is used for the treatment of some lymphomas. In patients with ALL it is often used to prevent the spread of cancer cells into the central nervous system.

Hematological cancers may sometimes be treated successfully by allogeneic bone marrow transplantation. The leukemic cells and the hematopoietic stem cells of the patient are completely eradicated by a combination of whole body irradiation and high dosages of chemotherapeutic agents. The patient then receives hematopoietic stem cells from a suitable donor to rebuild the patient's hematopoietic system. Nevertheless, despite careful genetic selection of the donor the transplanted leukocytes may attack cells of the host leading to graft-versus-host disease. This is a major risk associated with allogeneic bone marrow transplantation. Infection is another major risk and a significant cause of mortality after bone marrow transplantation, because the patient almost completely lacks white blood cells for several weeks after the transplantation and thus has no defense against pathogens.

The most commonly used—and in many instances the only available—systemic treatment for cancer is chemotherapy. For patients suffering from leukemia or metastases of solid tumors chemotherapy, thus, is the only treatment option. Chemotherapeutic agents are cytotoxic for all rapidly dividing cells. As cancer cells usually divide more rapidly than other cells in the body, they are preferably killed by these agents. Common groups of chemotherapeutic agents are substances that inhibit cell division by interfering with the formation of the mitotic spindle or agents which damage the DNA, e.g. by alkylating the bases. Because all rapidly dividing cells are targeted by chemotherapeutic agents, their side effects are usually severe. Depending on the substance used, they include organ toxicity (e.g. heart or kidney), immunosuppression, neurotoxicity and anemia. Some groups of chemotherapeutic agents, e.g. alkylating agents, even have the potential to cause cancer. Due to these side effects dosages have sometimes to be reduced or chemotherapy has to be discontinued completely. Furthermore, the side effects chemotherapy often prohibit the treatment of patients in bad general condition. Adding to all these problems is the often limited efficacy of chemotherapy. In some cases chemotherapy fails from the very beginning. In other cases tumor cells become resistant during the course of treatment. To combat the emergence of resistant tumor cells and to limit the side effects of chemotherapy combinations of different compounds with different modes of action are used. Nevertheless, the success of chemotherapy has been limited, especially in the treatment of solid tumors. However, in a few types of cancer, e.g. childhood ALL, the cure rates are relatively high (approximately 80%) (Pui and Evens, 2006, N. Engl. J. Med. 354: 166-178). For these cancers research focuses on means to reduce the undesired side effects without compromising the efficacy of the treatment.

Recently, drugs have become available whose mode of action is not based on toxicity against rapidly dividing cells. These compounds show a higher specificity for cancer cells and thus less side effects than conventional chemotherapeutic agents. Imatinib is used for the specific treatment of chronic myelogenous leukemia. This compound specifically inhibits an abnormal tyrosine kinase which is the product of a fusion gene of bcr and abl. Because this kinase does not occur in non-malignant cells, treatment with Imatinib has only mild side effects. However, Imatinib is not used for the treatment of hematological cancers other than myelogenous leukemia. Rituximab is a monoclonal antibody directed against the cluster of differentiation 20 (CD20), which is widely expressed on B-cells. It is used for the treatment of B cell lymphomas in combination with conventional chemotherapy.

Another important mode of action of chemotherapeutic agents is the induction of apoptosis. Many chemotherapeutic agents, e.g. alkylating agents, crosslinking agents or antimetabolites induce DNA damage which finally leads to apoptosis of the affected cells. The often poor efficacy of chemotherapeutic agents in tumor cells can be explained by the disruption of normal apoptotic pathways. Cells in many tumors, for instance, lack a functional copy of p53. The product of this gene is responsible for controlling the cell cycle and initiating DNA-repair in the case of DNA damage. In cells with large scale DNA damage p53 induces apoptosis. Without a functional p53 gene cells progress through the cell cycle and proliferate despite DNA-damage.

Apoptosis pathways involve diverse groups of molecules. One set of mediators implicated in apoptosis are so-called caspases, cysteine proteases that cleave their substrates specifically at aspartate residues. Caspases convey the apoptotic signal in a proteolytic cascade, with caspases cleaving and activating other caspases which subsequently degrade a number of target death proteins, such as poly (ADP-ribose) polymerase, eventually resulting in cell death. If one or more steps in this cascade are inhibited in tumor cells, these cells fail to undergo apoptosis and, thus, continue to grow. Caspase activation itself can be triggered by external stimuli affecting certain cell surface receptors, known to the person skilled in the art as so-called death receptors. Known death receptors mediating apoptosis after reception of an extrinsic signal include members of the tumor necrosis factor (TNF) receptor superfamily such as CD95 (APO-1/Fas) or TRAIL (TNF-related apoptosis inducing ligand) receptors 1 and 2. Stimulation of the death receptor CD95 leads to the formation of a cell membrane death inducing signaling complex (DISC, comprising CD95, FADD, pro-caspase 8 and c-FLIP) and among others, to the activation of caspase-8, which in turn activates other caspases and members of another group of apoptosis mediators.

In an alternative pathway of apoptosis induction known as intrinsic pathway of apoptosis induction, apoptosis is induced by an intracellular stress response via the mitochondria leading to the release of mitochondrial proteins. Extensive DNA damage is one of the factors that activate the intrinsic apoptotic pathway. Several Bcl-2 family members, commonly referred to as anti-apoptotic members of the Bcl-2 family, are thought to inhibit the release of the mitochondrial proteins and, thus, prevent cells from undergoing apoptosis.

Consequently, over-expression of the anti-apoptotic Bcl-2 family proteins Bcl-2, Bcl-xL, Bcl-w, and Mcl-1 (myeloid cell leukemia 1 protein) are frequently associated with tumor initiation, progression and resistance to conventional chemotherapies (Giam et al., 2009, Oncogene 27 Suppl 1:S128-36). These anti-apoptotic Bcl-2 proteins bind to the pro-apoptotic proteins Bak (Bcl-2 antagonist/killer) and Bax (Bcl-2-associated X protein) to prevent cell death. Only when Bak and Bax are released from their anti-apoptotic counterparts, they cause cell death by inducing the release of cytochrome c and activation of caspase-9 and -3. Thus, the anti-apoptotic proteins of the Bcl-2 family are validated drug targets for cancer treatment (Lessene et al., 2008, Nat Rev Drug Discov 7:989-1000). However, some tumor entities tend to overcome sensitivity to Bcl-2 family inhibitors by overproducing one or more anti-apoptotic Bcl-2 proteins, frequently Mcl-1, or by overproducing the X-linked inhibitor of apoptosis (XIAP), which prevents apoptosis at the effector phase by binding to and inhibiting activated caspase-3 and caspase-9, i.e. downstream of the anti-apoptotic Bcl-2 proteins.

ABT-737 and the orally bio-available ABT-263 (Navitoclax®) are small-molecule mimetics of the Bcl-2 homology domain 3, which inhibit Bcl-2, Bcl-xL and Bcl-w with high affinities. Both, ABT-737 and ABT-263, have been shown to be effective as single agent in hematological malignancies and also in other type of cancers (Lessene et al., 2008, Nat Rev Drug Discov 7:989-1000) Currently, ABT-263 is being investigated in clinical trials in patients with lymphoid malignancies and small cell lung cancer. Despite promising results obtained, a major drawback of the ABT-737/263-based cancer therapy is that tumor cells expressing high levels of Mcl-1 frequently resist treatment by these inhibitors (Konopleva et al., 2006, Cancer Cell 10:375-88; Tahir et al., 2007, Cancer Res 67:1176-83; van Delft et al., 2006, Cancer Cell 10:389-99; Chen et al., 2007, Cancer Res 67:782-91). In addition, sensitive cancer cells may develop acquired resistance due to selective up-regulation of Mcl-1 expression during long-term treatment as reported recently in the case of ABT-737-treated lymphomas (Yecies et al., 2010, Blood 115:3304-13). Furthermore, inhibition of Bcl-x_{L} by ABT-737/263 induces a concentration-dependent decrease in the number of circulating platelets (Tse et al., 2008, Cancer Res 68:3421-8). This side effect limits the ability to increase drug concentrations into a higher efficacious range.

Rocaglamide belongs to the group of chemical compounds characterized by a cyclopenta[b]benzofuran structure, said group also being referred to as flavaglines. Rocaglamide and rocaglamide derivatives can be isolated from Aglaia Species. It has been demonstrated that they possess antiproliferative activity (see e.g. U.S. Pat. No. 4,539,414; Dhar et al., 1973 Indian J Exp Vol. 11: 43-54; King et al., 1982 J Chem Soc Chem Comm Vol. 20: 1150-1151; Lee et al., 1998 Chem Biol Interact Vol. 115: 215-228; Bohnenstengel et al., 1999, Z. Naturforsch [C]. Vol. 54: 55-60; Bohnenstaengel et al., 1999 Z Naturforsch [C] Vol 54: 1075-1083; Kim et al., 2006 Anticancer Agents Med Chem Vol. 6: 319-345).

Rocaglamide derivatives have been shown to have an inhibitory effect on growth of a murine leukemia cell line (P-388), a mouse lymphoma cell line (RMA), a human breast cancer cell line (BC1), as well as primary tumor cells from acute myeloid leukemia (AML) patient samples in vitro and also in vivo (Hwang et al., 204, J. Org. Chem. 69:3350-3358; Lee et al., 1998, Chem. Biol. Interact 115: 215-228, Zhu et al. (2007), Int J Cancer 121(8): 1839; Zhu et al. (2009), Cell Death Differ 16(9): 1289). Rocaglamide has also been proposed to enhance the effect of compounds inducing the extrinsic pathway of apoptosis in cancer cells (WO 2010/057981).

Wogonin (5,7-dihydroxy-8-methoxy-2-phenyl-4H-chromen-4-one) can be prepared by extraction from roots of Scutellaria baicalensis Georgi or by chemical synthesis, e.g. by cyclization of 1,3-diaryl-diketons or by Wessely-Moser rearrangement. Wogonin has been shown to have anti-oxidant, anti-viral, anti-thrombotic and anti-inflammatory activities. The compound also shows cytostatic and pro-apoptotic effects on several tumor cells (e.g. US 20130059907). Wogonin and structurally related natural flavones are inhibitors of cyclin-dependent kinase 9 (CDK9).

Thus, there is a need in the art for improved cancer treatments, in particular treatments overcoming or preventing resistance of cancer cells to Bcl-2 family inhibitors. The problem underlying the present invention, thus, could be seen to provide means and methods complying with the aforementioned needs. The problem is solved by the embodiments of the present invention.

Accordingly, the present invention relates to a combined preparation for simultaneous, separate or sequential use comprising a) at least one flavagline or a pharmaceutically acceptable salt thereof; and/or at least one flavone comprising a 5-hydroxy-2-phenyl-4H-chromen-4-one (5-hydroxy-flavone) structure or a pharmaceutically acceptable salt thereof; and b) at least one agent activating the intrinsic pathway of apoptosis for use in the treatment of cancer.

As used herein, the term “preparation” relates to a pharmaceutical mixture of compounds, comprising at least two of the active compounds of the present invention as specified herein below and in the claims. The skilled person understands that the preparation according to the present invention may also comprise further compounds, e.g., one or more further pharmacologically active substances and/or, more preferably, pharmacologically acceptable carriers and/or auxiliary agents.

The terms “active compound” or “pharmaceutically active compound”, as used herein, relate to a compound according to the present invention mediating or causing a pharmaceutical effect in a cell and/or in a subject, as opposed to pharmaceutically inactive compounds, such as compounds included to improve galenic properties of a preparation. Preferably, the active compound or compounds is/are selected from the list consisting of a flavagline, a flavone comprising a 5-hydroxy-2-phenyl-4H-chromen-4-one (5-hydroxy-flavone) structure, and an agent activating the intrinsic pathway of apoptosis, as described herein, respectively. Some of the active compounds of the invention and/or salts or esters thereof will exist in different stereoisomeric forms; all of these forms are included in the present invention, provided they are pharmaceutically active. Preferably, the term active compound as used herein relates to those stereoisomers having the activity as described herein for the respective active compound. Most preferably, the term active compound relates to a compound having the conformation as described herein.

The term “combined preparation”, as used in this specification, relates to a preparation comprising the active compounds of the present invention for combined use. Thus, preferably, the combined preparation according to this specification is a preparation adapted such that the active compounds comprised therein are present in the body of a subject at an effective concentration for a certain time frame. More preferably, the active compounds are present in the body of a subject at an effective concentration sequentially or with overlapping time frames. Most preferably, the active compounds are present in the body of a subject at an effective concentration simultaneously for at least 50% of the treatment period, for at least 70% of the treatment period, or at least 90% of the treatment period.

Preferably, the combined preparation is for simultaneous use, i.e., preferably, the combined preparation comprises the active compounds adjusted in dose and/or pharmaceutical form for combined use at the same time. More preferably, the combined preparation for simultaneous use comprises all pharmaceutically active compounds in one preparation so that all compounds are administered simultaneously and in the same way.

Also preferably, the combined preparation is for separate use, i.e., preferably, the combined preparation comprises at least two physically separated preparations for separate administration, wherein each preparation contains at least one pharmaceutically active compound. The embodiment comprising separate preparations is preferred in cases where the pharmaceutically active compounds of the combined preparation have to be administered by different routes, e.g. parenterally and orally, due to their chemical or physiological properties, or in cases where the active compounds are chemically incompatible. Preferably, the at least two separated preparations are administered simultaneously. This means that the time frames of the administration of the preparations overlap.

Also preferably, the combined preparation is for sequential use, i.e., preferably, the combined preparation is for sequential administration of at least two preparations, wherein each preparation contains at least one pharmaceutically active compound. In that case, the administration of the single preparations shall occur in time frames which do not overlap so that the at least to pharmaceutically active compounds of the preparations are present in such plasma concentrations which enable the synergistic effect of the present invention. Preferably, the at least two preparations are administered in a time interval of 1 minute, 5 minutes, 15 minutes, 30 minutes, 1 hour, 2 hours, 4 hours, 8 hours, 16 hours, 1 day or 2 days. The embodiment of a preparation for sequential use is preferred in cases where the active compounds are of low physiological compatibility, e.g. because of an increase of adverse effects if taken simultaneously. Said embodiment is also preferred in cases where modes required modes of administration are temporally incompatible, e.g. in cases where one active compound is preferably administered before sleep, whereas the other is preferably administered in the morning.

The term “flavagline”, as used herein, relates to a chemical compound comprising a cyclopenta[b]benzofuran skeleton, preferably a cyclopenta[b]tetrahydroxy-benzofuran. More preferably, the term relates to cyclopenta[b]tetrahydroxy-benzofuranes produced by or extractable from a plant from the genus Aglaia (family Meliaceae). As used in this specification, said terms include derivatives of the said compounds as described herein above and below.

Preferably, the term flavagline relates to a compound of the formula (I)

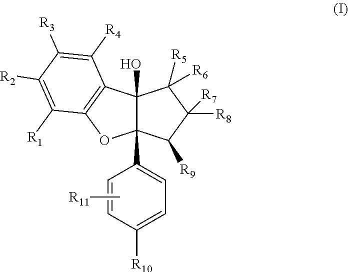

more preferably of the formula (X)

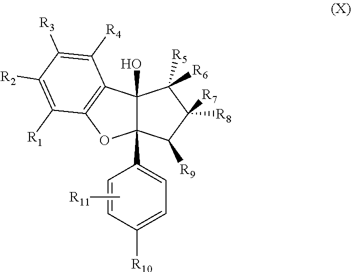

wherein

R₁ is selected from —H, halogen and alkyl;

R₂ is selected from alkoxy, halogen, and alkyl;

R₃ is selected from —H, halogen and alkyl;
or R₂ and R₃ together form a —O(CH₂)ₙO— unit, with n=1 or 2;

R₄ is selected from alkoxy, halogen, and alkyl;

R₅ is selected from hydroxyl, acyloxy, amino, monoalkylamino, dialkylamino and —NR₁₂—CHR₁₃—COOR₁₄, with
R₁₂ being selected from —H and alkyl,
R₁₃ being selected from phenyl and benzyl, which both may carry a substituent from the group hydroxyl, indolyl and imidazolylmethyl, and alkyl which may be substituted by a group selected from —OH, —SH, alkoxy, thioalkoxy, amino, monoalkylamino, dialkylamino, carboxy, carboxyalkyl, carboxamide and guanidino groups;
or R₁₂ and R₁₃ together form a —(CH₂)₃— or —(CH₂)₄— group;
R₁₄ being selected from alkyl and benzyl; in which case R₆ is hydrogen,

R₆ is selected from —H, halogen and alkyl;
or R5 and R6 together form an oxo or hydroxyimino group;

R₇ is —H;

R₈ is selected from —CONR₁₆R₁₇, —H, and —COOR₁₅ wherein
R₁₅ and R₁₆ are independently selected from methyl and —H, and
R₁₇ is selected from methyl, —H, 4-hydroxybutyl and 2-tetrahydrofuryl;

R₉ is selected from phenyl which is optionally substituted, and hetaryl which is optionally substituted;

R₁₀ is selected from alkoxy, —H, halogen, and alkyl, and

R₁₁ is selected from —H, hydroxyl, halogen, alkoxy and alkyl;
or R₁₀ and R₁₁ are in ortho-position to each other and together form a —O(CH₂)ₙO— unit, with n=1 or 2.

The term “alkyl”, as mentioned in the above definitions of the substituents R₁ to R₁₇, in each case refers to a substituted or an unsubstituted, linear or branched, acyclic or cyclic alkyl group, preferably an unsubstituted linear or branched acyclic alkyl group. More preferably, the term “alkyl”, as mentioned in the above definitions of the substituents R₁ to R₁₇, in each case preferably refers to a C₁-to C₄-alkyl group, namely methyl, ethyl, i-propyl, n-propyl, n-butyl, i-butyl, sec-butyl or tert-butyl. The above also applies when “alkyl” is used in “alkylamino” and “dialkylamino” and other terms containing the term “alkyl”.

The term “alkoxy”, as mentioned in the above definitions of the substituents R₁ to R₁₇, in each case refers to a substituted or an unsubstituted linear or branched, acyclic or cyclic alkoxy group, preferably an unsubstituted linear or branched acyclic alkoxy group. More preferably, the term “alkoxy”, as mentioned in the above definitions of the substituents R₁ to R₁₇, in each case preferably refers to a C₁-to C₄-alkoxy group, namely methoxy, ethoxy, i-propyloxy, n-propyloxy, n-butyloxy, i-butyloxy, sec-butyloxy or tert-butyloxy. The above also applies when “alkoxy” is used in “thioalkoxy” and other terms containing the term “alkoxy”.

The term “acyloxy”, as mentioned in the above definitions of the substituents R₁ to R₁₇, in each case refers to a substituted or an unsubstituted linear or branched, acyclic or cyclic acyloxy group, preferably an unsubstituted linear or branched acyclic acyloxy group. More preferably, the term “acyloxy”, as mentioned in the above definitions of the substituents R₁ to R₁₇, in each case preferably refers to a C₁-to C₄-acyloxy group, namely formyloxy, acetoxy, i-propyloxy, n-propyloxy, n-butyloxy, i-butyloxy, sec-butyloxy or tert-butyloxy.

The term “hetaryl” as used in the above definition refers to a 5-,6- or 7-membered carbocyclic saturated or non-saturated, aromatic or non-aromatic ring which may carry in the ring one or more heteroatoms from the group O, S, P, N.

The term “halogen” is known to the skilled person and preferably includes pseudhalogens; more preferably, the term relates to —F, —Cl, —Br, —I, —CN, or —SCN. Most preferably, the term relates to —Cl or —Br.

It is understood by the skilled person that formula (I) includes compounds wherein R6 is orientated above the plane of view and R5 then is orientated below the plane of view or vice versa. The same is true for R7 and R8 in formula (I), whereas in formula (X), R5 and R8 are orientated below the plane of view and R6 and R7 are orientated above the plane of view.

In a preferred embodiment of the present invention, the substituents R₁ to R₁₄ in formulae (I) and (X) have the following meanings:

R₁ and R₃ each are —H;

R₂ and R₄ each are independently selected from methoxy which is optionally substituted;

R₅ is selected from hydroxy, formyloxy and acetyloxy, alkylamino, —NR₁₂—CHR₁₃—COOR₁₄, with R₁₂ being selected from —H and alkyl,
R₁₃ being selected from: alkyl which may be substituted by —OH, —SH, alkoxy; thioalkoxy, amino, alkylamino, carboxy, carboxyalkyl, carboxamide and/or guanidino groups; and phenyl and benzyl, which both may carry a substituent from the group hydroxy, indolyl and imidazolylmethyl;
R₁₄ being selected from alkyl and benzyl;

R₆ is —H;

R₇ is —H;

R₈ is selected from —H, —COOCH₃, and —CONR₂₆R₂₇, with R₂₆R₂₇ being independently selected from alkyl and cycloalkyl, which may be substituted, preferably —CON(CH₃)₂;

R₉ is phenyl which is optionally substituted;

R₁₀ is methoxy;

R₁₁ is selected from —H and hydroxy,
or R₁₀ and R₁₁ are in ortho-position to each other and together form a —O(CH₂)ₙO— unit, with n =1 or 2.

In a still more preferred embodiment of the present invention, the flavagline relates to those of formula (I) or formula (X), wherein

R₁ and R₃ each are —H,

R₂ and R₄ each are optionally substituted methoxy,

R₅ is hydroxy or —NR₁₂—CHR₁₃—COOR₁₄,

with R₁₂ being selected from —H and alkyl,
R₁₃ being selected from: alkyl which may be substituted by —OH, —SH, alkoxy; thioalkoxy, amino, alkylamino, carboxy, carboxyalkyl, carboxamide and/or guanidino groups; and phenyl and benzyl, which both may carry a substituent from the group hydroxy, indolyl and imidazolylmethyl;
R₁₄ being selected from alkyl and benzyl;

R₆ and R₇ each are —H,

R₈ —CON(CH₃)₂;

R₉ is optionally substituted phenyl,

R₁₀ is methoxy and

R₁₁ is —H; or wherein

R₁ and R₃ each are —H,

R₂ and R₄ each optionally substituted methoxy,

R₅ is acetoxy or —NR₁₂—CHR₁₃—COOR₁₄,

with R₁₂ being selected from —H and alkyl,
R₁₃ being selected from: alkyl which may be substituted by —OH, —SH, alkoxy; thioalkoxy, amino, alkylamino, carboxy, carboxyalkyl, carboxamide and/or guanidino groups; and phenyl and benzyl, which both may carry a substituent from the group hydroxy, indolyl and imidazolylmethyl;
R₁₄ being selected from alkyl and benzyl;

R₆ and R₇ each are —H,

R₈ is —CON(CH₃)₂,

R₉ is optionally substituted phenyl,

R₁₀ is methoxy and

R₁₁ is —H; or wherein

R₁ and R₃ each are —H,

R₂ and R₄ each optionally substituted methoxy,

R₅ is formyloxy or —NR₁₂—CHR₁₃—COOR₁₄,

with R₁₂ being selected from —H and alkyl,
R₁₃ being selected from: alkyl which may be substituted by —OH, —SH, alkoxy; thioalkoxy, amino, monoalkylamino, dialkylamino, carboxy, carboxyalkyl, carboxamide and/or guanidino groups; and phenyl and benzyl, which both may carry a substituent from the group hydroxy, indolyl and imidazolylmethyl;
R₁₄ being selected from alkyl and benzyl;

R₆ and R₇ each are —H,

R₈ is —H or —COOCH₃,

R₉ is optionally substituted phenyl, and

R₁₀ and R₁₁ are in ortho-position to each other and together form a —O(CH₂)ₙO— unit, with n=1 or 2.

In a further embodiment of the present invention, R₈ is a group of the formula (c)

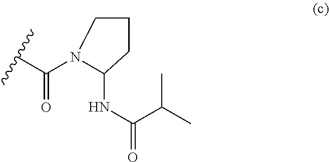 [SMILES: *C(=O)N1CCCC1NC(=O)C(C)C]

In still a further embodiment of the present invention, R₅ and R₈ together form a group of the formulae (a) or (b)

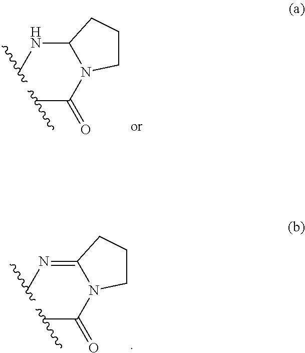 [SMILES: *NC1CCCN1C(*)=O ; */N=C1/CCCN1C(*)=O]

Preferably, the term flavagline relates to a compound selected from the group consisting of rocaglamide, aglaroxin C, cyclorocaglamide, rocaglaol, methylrocaglate (aglafolin), desmethylrocaglamide, pannellin and the recently isolated dioxanyloxy-modified derivatives silvestrol and episilvestrol (Hwang et al., 2004, J. Org. Chem. Vol. 69: pages 3350-3358). It is understood by the skilled person that the term “rocaglamide” is a generic term for a compound of formula (II) (named Rocaglamide A or Roc-A in the example section), formula (III), formula (IV), formula (V) (named Rocaglamide Q or Roc-Q in the example section), formula (VI) (referred to as Rocaglamide AR or Roc-AR in the present application), formula (VII) (known as Rocaglamide U or Roc-U), and formula (VIII) (known as Rocaglamide W or Roc-W). More preferably, the flavagline is Rocaglamide Q or Rocaglamide AR; most preferably, the flavagline is Rocaglamide A ((1R,2R,3S,3aR,8bS)-1,8b-dihydroxy-6,8-dimethoxy-3a-(4-methoxyphenyl)—N,N-dimethyl-3-phenyl-2,3-dihydro-1H-cyclopenta[b][1]benzofuran-2-carboxamide).

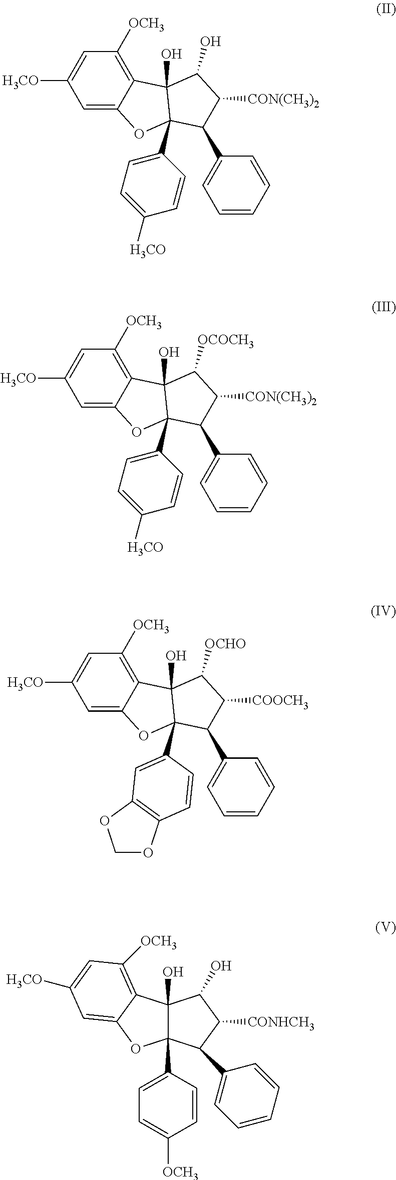

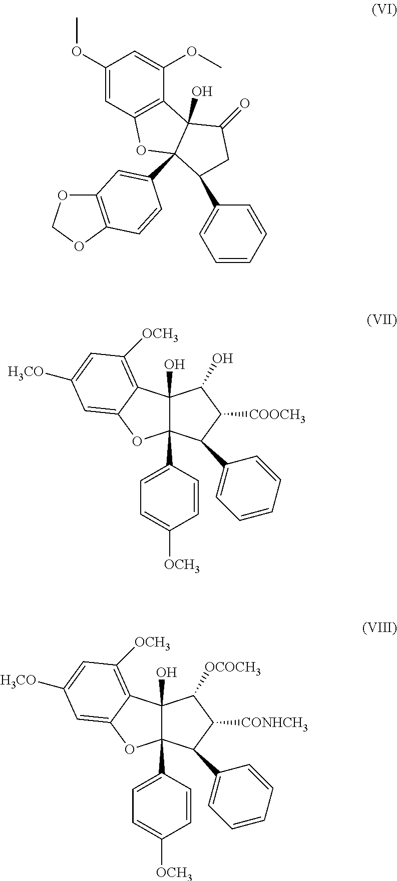

In a preferred embodiment, the term flavagline relates to a compound selected from the group consisting of rocaglamide, aglaroxin C, cyclorocaglamide, rocaglaol, methylrocaglate (aglafolin), desmethylrocaglamide, and pannellin. Thus, in preferred embodiments, the term flavagline does not relate to silvestrol and/or episilvestrol.

For the preparation of the rocaglamide derivatives according to the present invention, reference is made to WO 00/07579, WO 03/045375 and WO 00/08007.

As used herein, the term “pharmaceutically acceptable salt” refers to those salts which are, within the scope of sound medical judgment, suitable for use in contact with the tissues of humans and animals without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66: 1-19 (1977). The salts can be prepared in situ during the final isolation and purification of the rocaglamide derivatives, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, arginine, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

The term “derivative”, as used herein, is known to the skilled person and relates to a compound obtainable from an active compound according to the present invention by chemical modification in, preferably, at most three chemical modification reactions, more preferably, in at most two chemical modification reactions, or, most preferably, in one chemical modification reaction. Preferably, the derivative comprises the same structural skeleton as the parent compound as described herein above and below. More preferably, the derivative has the same or a similar activity with regard to the diseases referred to herein as the parent compound as described herein above and below; or, also preferably, the derivative is an inactive precursor which is metabolized by the metabolism of the subject treated with said derivative into an active compound having the same or a similar activity with regard to the diseases referred to herein as the parent compound as described herein above and below. Preferred derivatives are compounds obtained from the compounds of the present invention by alkylation, preferably methylation or ethylation, acylation, preferably acetylation, glycosylation, hydroxylation, deacylation or demethylation, or derivatization with a piperazine, piperidine, piperidinamine, teneraic acid, piperidinepropanol, halogen, preferably F or Cl, more preferably I or Br, amino acid, or polypeptide, preferably olipopeptide, functional group.

As used herein, the term “flavone” is used in its usual chemical meaning and relates to a compound structurally characterized as comprising a 2-phenyl-4H-chromen-4-one skeleton. Consequently, the term “5-hydroxy-flavone” relates to a compound structurally characterized as comprising a 5-hydroxy-2-phenyl-4H-chromen-4-one skeleton. As used in this specification, said terms include derivatives of the said compounds. Thus, preferably, the term “5-hydroxy-flavone” relates to a compound of the formula (IX)

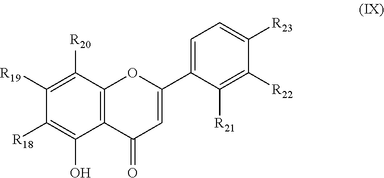

wherein

R₁₈ is —H, —OH, —CH₃, —CH₂OH, —OCH₃, phenyl, or hydroxyl-substituted phenyl,

R₁₉ is —OH, —H, —CH₃, —CH₂OH, —OCH₃, phenyl, or hydroxyl-substituted phenyl,

R₂₀ is —OCH₃, —H, or a heterocyclic group (P)

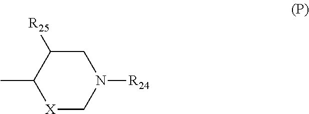

wherein X is —CH₂—, —O—, or —CH(OH)—, R₂₄ is —H, —CH₃, or —OCH₃, R₂₅ is —H or —OH,

R₂₁ is —H, —OH, —OCH₃, or —NH₂,

R₂₂ is —H, —OH, or —OCH₃, and

R₂₃ is —H or —OH, or —OCH₃.

More preferably, the 5-hydroxy-flavone is a compound of formula (IX), wherein R₁₉ is —OH and the other substituents are as defined above. Even more preferably, the 5-hydroxy-flavone is a compound of formula (IX), wherein R₁₉ is —OH, R₂₀ is —OCH₃ or —OH, and the other substituents are as defined above.

Particularly preferred are embodiments wherein R₁₉ is —OH and R₂₁ is —H, and
R₂₀, R₂₂, R₂₃ are —H, R₁₈ is —OH (Baicalein), or
R₂₀ and R₂₂ are —H, R₂₃ is —OH, and R₁₈ is —H (Apigenin), or
R₂₀, R₂₂, R₂₃, and R₁₈ are —H (Chrysin), or
R₂₀ is —H, R₂₂, R₂₃ are —OH, and R₁₈ is —H (Luteolin).

Most preferably, the 5-hydroxy-flavone is a compound of formula (IX), wherein R₁₈ is —H, R₁₉ is —OH, R₂₀ is —OCH₃, and R₂₁, R₂₂, and R₂₃ are —H (Wogonin).

Preferably, the 5-hydroxy-flavone comprises a 5,7-dihydroxy-flavone skeleton. More preferably, the 5-hydroxy-flavone comprises a 5,7,8-trihydroxy-flavone skeleton or a 5,7-dihydroxy-8-methoxy-flavone skeleton. Most preferably, the 5-hydroxy-flavone is 5,7-Dihydroxy-8-methoxy-2-phenyl-4H-chromen-4-one, known to the skilled person as Wogonin. The term “agent activating the intrinsic pathway of apoptosis”, as used herein, relates to a chemical compound modulating the intrinsic pathway of apoptosis in a way that a cell contacted with said compound undergoes apoptosis, wherein said cell preferably is a cell insensitive to normal induction of apoptosis. The term “normal induction of apoptosis” is known to the skilled person and relates to any treatment or condition causing apoptosis to occur in a normal cell, preferably a non-tumor cell, most preferably in platelet cells, peripheral blood T cells, peripheral blood B cell, bone marrow stem cells, or in cardiac muscle cells. Preferably, the compound activating the intrinsic pathway of apoptosis is an inhibitor of the interaction of at least one, more preferably at least two, most preferably at least three anti-apoptotic members of the Bcl-2 family of proteins with its or their natural ligand or ligands. Preferably, said anti-apoptotic members of the Bcl-2 family of proteins are selected from the group consisting of Bcl-2, Bcl-x_{L}, and Bcl-w. More preferably, the compound activating the intrinsic pathway of apoptosis is a mimetic of the Bcl-2 homology domain 3 (BH3 domain). Such molecules and means of identifying them are known in the art and have been summarized, e.g. in Lessene et al., 2008, Nat Rev Drug Discovery 7: 989.

Preferably, the compound activating the intrinsic pathway of apoptosis has an IC₅₀, EC₅₀, or Kᵢ for at least one, more preferably at least two, of Bcl-2, Bcl-x_{L}, and Bcl-w at least 10 fold, more preferably at least 25 fold, most preferably at least 100 fold lower than the respective IC₅₀, EC₅₀, or Kᵢ value for Mcl-1.

Preferably, the compound activating the intrinsic pathway of apoptosis is selected from the list consisting of a stapled peptide derived from a Bcl-2-interacting protein, in particular SAHBA, which is a stapled peptide derived from Bcl-2-interacting mediator of cell death (Walensky et al., 2004, Science 305: 1466); a Terphenyl derivative (Yin et al., 2005, J Am Chem Soc. 127(291. 101911 in narticular the derivative of the formula (XI)

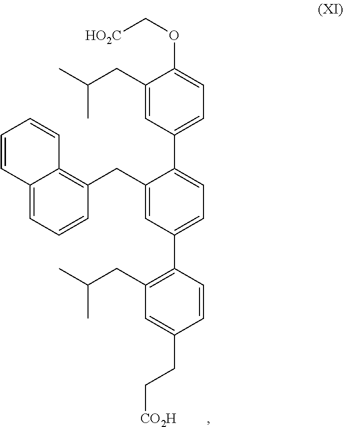 [SMILES: CC(C)Cc1cc(-c2ccc(-c3ccc(CCC(=O)O)cc3CC(C)C)cc2Cc2cccc3ccccc23)ccc1OCC(=O)O]

a Benzoylurea derivative (US 2008/153802), an Isooxazolidine (WO 2008/060569), and A-385358 (Wendt et al., 2006, J Med Chem 49:1165). More preferably, the compound activating the intrinsic pathway of apoptosis is selected from the list consisting of ABT-263 ((R)-4-(4-((4′-chloro -4,4-dimethyl-3,4,5,6-tetrahydro-[1,1′-biphenyl]-2-yl)methyl)piperazin-1-yl)--N-((4-((4-morpholino -1-(phenylthio)butan-2-yl)amino)-3-((trifluoromethyl)sulfonyl)phenyl)sulfonyl)benzamide), ABT-737 (4-[4-[(4′-chloro[1,1′-biphenyl]-2-yl)methyl]-1-piperazinyl]-N-[[4-[[1R)-3-(dimethylamino)-1-[(phenylthio)methyl]propyl]amino]-3-nitrophenyl]sulfonyl]-Benzamide), and ABT-199 (4-[4-[[2-(4-chlorophenyl)-4,4-dimethyl-1-cyclohexen-1-yl]methyl]-1-piperazinyl]-N-[[3-nitro-4-[[(tetrahydro -2H-pyran-4-yl)methyl]amino]phenyl]sulfonyl]-2-(1H-pyrrolo[2,3-b]pyridin-5-yloxy)-Benzamide.

Compounds activating the intrinsic pathway of apoptosis have been known to have the potential to cause severe side effects, e.g., by inducing apoptosis in non-malignant cells. This effect is relevant in particular at high doses. Thus, according to the present invention, preferably, the compound activating the intrinsic pathway of apoptosis is used at a concentration avoiding severe side effects. More preferably, said severe side effects include thrombocytopenia.

The term “treatment” refers to an amelioration of the diseases or disorders referred to herein or the symptoms accompanied therewith to a significant extent. Said treating as used herein also includes an entire restoration of the health with respect to the diseases or disorders referred to herein. It is to be understood that treating as used in accordance with the present invention may not be effective in all subjects to be treated. However, the term shall require that a statistically significant portion of subjects suffering from a disease or disorder referred to herein can be successfully treated. Whether a portion is statistically significant can be determined without further ado by the person skilled in the art using various well known statistic evaluation tools, e.g., determination of confidence intervals, p-value determination, Student's t-test, Mann-Whitney test etc. Preferred confidence intervals are at least 90%, at least 95%, at least 97%, at least 98% or at least 99%. The p-values are, preferably, 0.1, 0.05, 0.01, 0.005, or 0.0001. Preferably, the treatment shall be effective for at least 60%, at least 70%, at least 80%, or at least 90% of the subjects of a given cohort or population.

The term “cancer”, as used herein, relates to a disease of an animal, including man, characterized by uncontrolled growth by a group of body cells (“cancer cells”). This uncontrolled growth may be accompanied by intrusion into and destruction of surrounding tissue and possibly spread of cancer cells to other locations in the body.

Preferably, the cancer is selected from the list consisting of acute lymphoblastic leukemia, acute myeloid leukemia, adrenocortical carcinoma, aids-related lymphoma, anal cancer, appendix cancer, astrocytoma, atypical teratoid, basal cell carcinoma, bile duct cancer, bladder cancer, brain stem glioma, breast cancer, burkitt lymphoma, carcinoid tumor, cerebellar astrocytoma, cervical cancer, chordoma, chronic lymphocytic leukemia, chronic myelogenous leukemia, colon cancer, colorectal cancer, craniopharyngioma, endometrial cancer, ependymoblastoma, ependymoma, esophageal cancer, extracranial germ cell tumor, extragonadal germ cell tumor, extrahepatic bile duct cancer, gallbladder cancer, gastric cancer, gastrointestinal stromal tumor, gestational trophoblastic tumor, hairy cell leukemia, head and neck cancer, hepatocellular cancer, hodgkin lymphoma, hypopharyngeal cancer, hypothalamic and visual pathway glioma, intraocular melanoma, kaposi sarcoma, laryngeal cancer, medulloblastoma, medulloepithelioma, melanoma, merkel cell carcinoma, mesothelioma, mouth cancer, multiple endocrine neoplasia syndrome, multiple myeloma, mycosis fungoides, nasal cavity and paranasal sinus cancer, nasopharyngeal cancer, neuroblastoma, non-hodgkin lymphoma, non-small cell lung cancer, oral cancer, oropharyngeal cancer, osteosarcoma, ovarian cancer, ovarian epithelial cancer, ovarian germ cell tumor, ovarian low malignant potential tumor, pancreatic cancer, papillomatosis, paranasal sinus and nasal cavity cancer, parathyroid cancer, penile cancer, pharyngeal cancer, pheochromocytoma, pituitary tumor, pleuropulmonary blastoma, primary central nervous system lymphoma, prostate cancer, rectal cancer, renal cell cancer, retinoblastoma, rhabdomyosarcoma, salivary gland cancer, sezary syndrome, small cell lung cancer, small intestine cancer, soft tissue sarcoma, squamous cell carcinoma, squamous neck cancer, testicular cancer, throat cancer, thymic carcinoma, thymoma, thyroid cancer, urethral cancer, uterine sarcoma, vaginal cancer, vulvar cancer, waldenstrom macroglobulinemia, and wilms tumor. More preferably, the cancer is leukemia, lymphoma, particularly non—Hodgkin lymphoma, small cell lung cancer, or breast cancer, in particular estrogen receptor-positive breast cancer.

Preferably, the cancer is a cancer insensitive to treatment with an agent activating the intrinsic pathway of apoptosis as defined herein above. More preferably, the cancer is a cancer insensitive to an inhibitor of Bcl-2, and/or Bcl-x_{L} and/or Bcl-w. Most preferably, the cancer is a cancer overproducing Mcl-1, in particular overproducing Mcl-1 but not an inhibitor of apoptosis acting downstream of the anti-apoptotic members of the Bcl-2 family of proteins (e.g., preferably, X-linked inhibitor of apoptosis (XIAP)). Preferably, the cancer is a cancer intrinsically insensitive to an agent activating the intrinsic pathway of apoptosis; more preferably, the cancer is a primary cancer or a metastasis thereof overproducing Mcl-1, in particular overproducing Mcl-1 but not an inhibitor of apoptosis acting downstream of the anti-apoptotic members of the Bcl-2 family of proteins (e.g., preferably, XIAP). Even more preferably, the cancer is a cancer insensitive to treatment with an agent activating the intrinsic pathway of apoptosis after a subject was treated with an agent activating the intrinsic pathway of apoptosis, i.e., a residual cancer or relapse. Most preferably, the cancer is a residual cancer or relapse overproducing Mcl-1, in particular overproducing Mcl-1 but not an inhibitor of apoptosis acting downstream of the anti-apoptotic members of the Bcl-2 family of proteins (e.g., preferably, XIAP).

Advantageously, it was found in the work underlying the present invention that flavaglines and 5-hydroxy-flavones can sensitize cancer cells resistant to agents activating the intrinsic pathway of apoptosis. Thus, by using a combined therapy of an agent activating the intrinsic pathway of apoptosis with a flavagline and/or a 5-hydroxy-flavone, cancers resistant to single treatment are amenable to treatment again. Also, by using a combined treatment from the beginning, resistant mutants overproducing Mcl-1 can be avoided. Moreover, in cases where treatment with an agent activating the intrinsic pathway of apoptosis is desirable to be continued despite the cancer having become insensitive, treatment can be continued without a need to increase the dose of the agent activating the intrinsic pathway of apoptosis to an extent causing severe adverse effects, like thrombocytopenia, to occur.

The definitions made above apply mutatis mutandis to the following. Additional definitions and explanations made further below also apply for all embodiments described in this specification mutatis mutandis.

Preferably, the combined preparation for use in medicine or for treating cancer according to the present invention is provided in a medicament.

Thus, the present invention also relates to a medicament for the treatment of cancer which contains a) least one flavagline or a pharmaceutically acceptable salt thereof; and/or at least one 5-hydroxy-flavone or a pharmaceutically acceptable salt thereof; and b) at least one agent activating the intrinsic pathway of apoptosis, and at least one pharmaceutically acceptable carrier.

The term “medicament”, as used herein, relates to a pharmaceutical composition comprising or consisting of the active compounds of the present invention and optionally one or more pharmaceutically acceptable carrier. The active compounds of the present invention can be formulated as pharmaceutically acceptable salts as described herein above. The pharmaceutical compositions are, preferably, administered locally or topically, or, more preferably, systemically. Suitable routes of administration conventionally used for drug administration are oral, intravenous, or parenteral administration as well as inhalation. However, depending on the nature of an active compound and the disease to be treated, the pharmaceutical compositions may be administered by other routes as well. For example, peptides may be administered in a gene therapy approach by using viral vectors or viruses or liposomes.

Moreover, the active compounds can be administered in combination with other drugs either in a common pharmaceutical composition or as separated pharmaceutical compositions as described herein above. The active compounds are, preferably, administered in conventional dosage forms prepared by combining the drugs with standard pharmaceutical carriers according to conventional procedures. These procedures may involve mixing, granulating and compressing or dissolving the ingredients as appropriate to the desired preparation. It will be appreciated that the form and character of the pharmaceutically acceptable carrier or diluent is dictated by the amount of active ingredient with which it is to be combined, the route of administration and other well-known variables.

The carrier(s) must be acceptable in the sense of being compatible with the other ingredients of the formulation and being not deleterious to the recipient thereof. The pharmaceutical carrier employed may be, for example, either a solid, a gel or a liquid. Exemplary of solid carriers are lactose, terra alba, sucrose, talc, gelatin, agar, pectin, acacia, magnesium stearate, stearic acid and the like. Exemplary of liquid carriers are phosphate buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pa.

The diluent(s) is/are selected so as not to affect the biological activity of the active compounds. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

A therapeutically effective dose refers to an amount of the active compounds to be used in a pharmaceutical composition of the present invention, which prevents, ameliorates or treats the symptoms accompanying a disease or condition referred to in this specification. Therapeutic efficacy and toxicity of such active compounds can be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, LD₅₀/ED₅₀.

The dosage regimen will be determined by the attending physician and other clinical factors; preferably in accordance with any one of the above-described methods. As is well known in the medical arts, dosages for any one patient depends upon many factors, including the patient's size, body surface area, age, the particular active compound to be administered, sex, time and route of administration, general health, and other drugs being administered concurrently. Progress can be monitored by periodic assessment. A typical dose can be, for example, in the range of 1 to 1000 μg; however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. However, depending on the subject and the mode of administration, the quantity of substance administration may vary over a wide range to provide from about 0.01 mg per kg body mass to about 10 mg per kg body mass, preferably. The pharmaceutical compositions and formulations referred to herein are administered at least once in order to treat or ameliorate or prevent a disease or condition recited in this specification. However, the said pharmaceutical compositions may be administered more than one time, for example from one to four times daily up to a non-limited number of days. Specific pharmaceutical compositions are prepared in a manner well known in the pharmaceutical art and comprise at least one active compound referred to herein above in admixture or otherwise associated with a pharmaceutically acceptable carrier or diluent. For making those specific pharmaceutical compositions, the active compound will usually be mixed with a carrier or the diluent, or enclosed or encapsulated in a capsule, sachet, cachet, paper or other suitable containers or vehicles. The resulting formulations are to be adapted to the mode of administration, i.e. in the forms of tablets, capsules, suppositories, solutions, suspensions or the like. Dosage recommendations shall be indicated in the prescribers or users instructions in order to anticipate dose adjustments depending on the considered recipient. Moreover, the present invention relates to a kit comprising the combined preparation for use or a medicament according to the present invention and a means for administering at least one of its components.

The term “kit”, as used herein, refers to a collection of the aforementioned components, preferably, provided separately or within a single container. Examples for such components of the kit as well as methods for their use have been given in this specification. The kit, preferably, contains the aforementioned components in a ready-to-use formulation. The kit, preferably, comprises instructions for carrying out a method of the present invention. Also preferably, the kit may comprise instructions, e.g., a user's manual or a package leaflet for administering the combined preparation or the medicament with respect to the applications provided by the methods of the present invention. Details are to be found elsewhere in this specification. Additionally, such user's manual may provide instructions about correctly using the components of the kit. A user's manual may be provided in paper or electronic form, e.g., stored on CD or CD ROM. The present invention also relates to the use of said kit in any of the methods according to the present invention. The kit of the present invention, preferably comprises a means for administering at least one of its components. The skilled person knows that the selection of the means for administering depends on the properties of the compound to be administered and the way of administration. Where the compound is or is comprised in a liquid and the mode of administration is oral, said means, preferably, is a drinking aid, such as a spoon or a cup. In case the liquid shall be administered intravenously, the means for administering may be an i.v. equipment.

The present invention also relates to a method of inhibiting a cancer cell, comprising a) contacting said cancer cell with the combined preparation the present invention, and b) thereby inhibiting said cancer cell.

The method of the present invention, preferably, is an in vitro method. Moreover, it may comprise steps in addition to those explicitly mentioned above. For example, further steps may relate, e.g., to obtaining and/or culturing a cancer cell for step a). Moreover, one or more of said steps may be performed by automated equipment.

The term “cancer cell” has been defined herein above. Preferably, the cancer cell is a cell of an animal. More preferably, the cancer cell is a mammalian cell, even more preferably, a human cell, most preferably a human cancer cell overproducing Mcl-1, in particular overproducing Mcl-1 but not an inhibitor of apoptosis acting downstream of the anti-apoptotic members of the Bcl-2 family of proteins (e.g., preferably, XIAP).

The term “contacting” as used in the context of the method of inhibiting a cancer cell of the present invention is understood by the skilled person. Preferably, the term relates to bringing a combined preparation of the present invention into physical contact with a cancer cell and thereby allowing the combined preparation and the cancer cell to interact.

The term “inhibiting a cancer cell”, as used herein, relates to preventing a cancer cell from migrating and/or proliferating. Preferably, the term relates causing said cancer cell to undergo apoptosis. Thus, more preferably, the term relates to killing said cancer cell.

The present invention further relates to a method of treating cancer in a subject afflicted with cancer, comprising administering a combined preparation of the present invention to said subject, thereby treating cancer.

The method of treating cancer, preferably, is an in vivo method of treatment. Moreover, it may comprise steps in addition to those explicitly mentioned above. For example, further steps may relate, e.g., to diagnosing cancer in a subject. Moreover, one or more of said steps may be performed by automated equipment.

The term “subject afflicted with cancer”, as used herein, relates to an individual comprising at least one cancer cell of a cancer as defined herein above. Preferably, the subject is a mammal; more preferably, the subject is a human.

The present invention also relates to a use of a) at least one flavagline or a pharmaceutically acceptable salt thereof; and/or at least one 5-hydroxy-flavone or a pharmaceutically acceptable salt thereof; and b) at least one agent activating the intrinsic pathway of apoptosis for the manufacture of a medicament for treating cancer.

All references cited in this specification are herewith incorporated by reference with respect to their entire disclosure content and the disclosure content specifically mentioned in this specification.

### FIGURE LEGENDS

FIG. 1: Rocaglamide A (Roc-A) sensitizes cancer cells for the anticancer activity of ABT-263.

(A) sensitization of leukemic Jurkat T cells (J16). (B) sensitization of HTLV-1 infected MT-2 cells. (C) western blots of caspases and PARP in leukemic Jurkat T cells (J16) after treatment with ABT-263 and/or Rocaglamide A

FIG. 2: Wogonin (Wogo) enhances ABT-263-induced apoptosis in leukemic T cells.

(A) ABT-263 induces apoptotic cell death in leukemic T cell lines. Human leukemic T cell lines CEM, Jurkat and Molt-4 were treated with 1 μM ABT-263 for 48 h. Apoptotic cell death was determined by DNA fragmentation. Results are an average of three independent experiments. (B) and (C) Wogonin enhances the efficacy of ABT-263 in leukemic T cells. Jurkat and CEM cells were treated with different concentrations of wogonin in the presence or absence of indicated concentrations of ABT-263 (B) or CEM, Jurkat and Molt-4 T cells were treated with different concentrations of ABT-263 in the absence or presence of indicated concentrations of wogonin (C). Apoptotic cell death was determined by DNA fragmentation after 24 h treatment. Results are representative of three independent experiments each performed in duplicate assays. (D) Wogonin down-regulates Mcl-1 expression in leukemic T cells. CEM, Jurkat and Molt-4 were treated with 50 μM Wogonin for 8 h. Total cell lysates were subjected to Western blot analysis by indicated antibodies. Representative blots for triplicate experiments are presented.

FIG. 3: Wogonin potentiates the efficacy of ABT-263 in different types of tumor cells.

(A) Sensitivities of different types of malignant cells to ABT-263. CEM and additional 8 different types of cancer cell lines were compared for their sensitivities to ABT-263. All indicated cell lines were treated with 1 μM of ABT-263 for 48 h. Apoptotic cell death was determined by DNA fragmentation. Results are representative of three independent experiments. (B) Effect of wogonin on ABT-263-mediated apoptosis in different cell lines. The indicated cell lines were treated with different concentrations of ABT-263 in the absence or presence of 50 μM of wogonin. Apoptotic cell death was determined by DNA fragmentation after 48 h treatment. Results are representative of at least two independent experiments performed as triplicate assays. (C) Effect of wogonin on Mcl-1 expression in different types of tumor cell lines. Different tumor cell lines were treated with 50 μM of wogonin for 8 h. The expression levels of Mcl-1, Bcl-2, Bcl-xL and Bcl-w were examined by Western blot. Representative blots from two to three independent experiments are shown.

FIG. 4: Wogonin re-sensitizes tumor cells which have developed acquired resistance to ABT-263.

(A-D) Leukemic T cells developed acquired resistance to ABT-263 during long-term exposure. Jurkat and CEM cells were treated with increasing concentrations up to 10 μM of ABT-263 for Jurkat (3 months) and 1 μM ABT-263 for CEM (2 months). The sensitivities of the cells to ABT-263 were examined by the apoptosis assay as described before (A and C). The expression levels of Mcl-1, Bcl-2, Bcl-xL and Bcl-w were examined by Western blot analysis (B and D). (E and F) Wogonin re-sensitizes resistant cells towards ABT-263-induced apoptotic cell death. The ABT-263-resistant Jurkat (Jurkat-R) and CEM (CEM-R) were treated with ABT-263 in the absence or presence of wogonin. Apoptotic cell death was determined by DNA fragmentation and the expression levels of the Bcl-2 family proteins were analyzed by Western blot. Results are an average of three independent assays.

FIG. 5: The efficacy of ABT-263 can be enhanced by wogonin-related natural flavones

(A) Wogonin-related natural flavones enhance ABT-263-induced apoptosis in CEM cells. CEM leukemic T cells were treated with different concentrations of ABT-263 in the absence or presence of 10 μM of different flavones as indicated. After 30 h treatment, apoptotic cell death was determined by DNA fragmentation. Results are the average of two independent experiments. (B) Down-regulation of Mcl-1 expression by wogonin-related flavones. CEM cells were treated with 50 μM of different flavones as indicated for 8 h. Total cell lysates were examined by Western blot for the expression levels of indicated anti-apoptotic Bcl-2 family proteins. Representative blots from two independent experiments are shown.

FIG. 6: Wogonin does not enhance the toxicity of ABT-263 in proliferating normal T cells or peripheral platelets.

(A) Wogonin does not potentiate the toxicity of ABT-263 in proliferating normal T cells. Normal T cells were isolated from peripheral blood of healthy donors and activated to proliferate as described in the Materials and Methods. The proliferating T cells were treated with different concentrations of ABT-263 in the absence or presence of wogonin as indicated. Apoptotic cell death was determined by DNA fragmentation. Results are presented as pooled data from four independent donors. (B) Proliferating normal T cells express higher levels of Mcl-1 and Bcl-2 than leukemic Jurkat and Molt-4 T cells. Total cell lysates of activated T cells from three healthy donors were compared with leukemic cells for the expression levels of Mcl-1, Bcl-2, Bcl-xL and Bcl-w. CDK6 is known to be over-expressed in many tumor cells and was used as a marker for malignant cells. Representative blots from two independent experiments are shown. (C) Wogonin does not enhance the toxicity of ABT-263 to peripheral platelets. Human platelets in 10% FCS were treated with different concentrations of ABT-263 in the absence or presence of wogonin as indicated. After 1 h treatment, apoptotic cell death was determine by staining with annexin V/FITC. Results are average of two independent assays.

FIG. 7: In vivo study of combination treatment with ABT-263 and wogonin

(A) Significant inhibition in tumor growth by ABT-263 in combination with wogonin. After the tumors reached to approximate 35 mm³, tumor size matched mice were treated without or with ABT-263 (50 mg/kg) in the presence or absence of wogonin (50 mg/kg) at the days indicated. (B) The body weight at the end of the experiment. (C) Tumor samples.

FIG. 8: Wogonin sensitizes tumor cells to ABT-199.

(A) CEM, Jurkat 16 and Molt-4 cells were treated with increasing concentrations up to 10 μM of ABT-199. The sensitivities of the cells to ABT-263 were examined by the apoptosis assay as described before (FIG. 4). (B) Wogonin sensitizes cells towards ABT-199-induced apoptotic cell death. CEM, Jurkat 16 and Molt-4 cells were treated with ABT-199 in the absence or presence of the indicated concentrations of wogonin. Apoptotic cell death was determined by DNA fragmentation as described above. Results are an average of three independent assays.

The following Examples shall merely illustrate the invention. They shall not be construed, whatsoever, to limit the scope of the invention.

### EXAMPLE 1

### Materials and Methods Used for Obtaining the Results Underlying the Present Invention

Cell Lines and Culture

The human malignant cell lines used in this study are the T cell leukemic cell lines CEM, Molt-4 and Jurkat (J16), the HL cell line KM-H2, the cervix adenocarcinoma cell line HeLa, the melanoma cell line A375, the hepatocellular carcinoma cell line HepG2, the pancreatic carcinoma cell line MiaPaca, the breast cancer cell line MCF-7, the prostatic cancer cell line PC3 and the colon carcinoma cell line HCT116. All cells were cultured in RPMI 1640 or DMEM medium (GIBCO laboratories, Grand Island, USA), supplemented with 10% FCS, 100 Units/ml penicillin (GIBCO), 100 μg/m1 streptomycin (GIBCO) and 2 mM L-glutamine (GIBCO) at 37° C. and 5%.

### Generation of ABT-263-Resistant Cell Lines

To generate resistant leukemic cells, Jurkat and CEM cells were continuously treated with increasing concentrations of ABT-263. After the cells displayed a viability of approximately 90% and were able to grow at a rate equivalent to the parental line, drug concentrations were doubled until 10 μM ABT-263 for Jurkat and 1 μM ABT-263 for CEM.

Preparation of Human T Cells and Platelets from Peripheral Blood.

Human peripheral blood T cells were prepared as described previously 23 and were more than 90% CD3 positive. For generation of proliferating T cells, freshly isolated T cells were cultured at 2×106 cells/ml and were activated with 1 mg/ml PHA overnight. Activated T cells were then washed three times and cultured for additional 5 days in the presence of 25 U/ml IL-2. Platelets were prepared as described in Vogler et al., 2011, Blood 117: 7145-54.

Determination of Apoptosis

Wogonin (BIOTREND Chemicals AG, Wangen, Switzerland), apigenin, chrysin, luteolin and baicalein (Sigma, St. Louis, USA) were solved in dimethyl sulfoxide (DMSO; Roth, Karlsruhe, Germany) at a stock concentration of 50 mM. Cells were treated with different concentrations of different flavones or ABT-263 (Selleck Chemicals, Houston, USA) for 24 to 48 h. Apoptotic cell death was examined by analysis of DNA fragmentation as previously described 23 (Fas et al.,2006, Blood 108: 3700-6). Results are presented as % specific DNA fragmentation using the formula: (percentage of experimental apoptosis-percentage of spontaneous apoptosis)/(100-percentage of spontaneous apoptosis)×100.

### Western Blot Analysis

For each sample, 1×107 cells were lysed as previously described. 23 Equal amounts of protein were separated on 5-13% SDS-PAGE depending on the molecular sizes of the proteins and blotted onto a nitrocellulose membrane (Amersham Biosciences, Little Chalfon, UK) as previously described. 23 The following antibodies were used: Bad, Bax, Bcl-xL, Bcl-w, XIAP from Cell Signaling Technology, Danvers, USA; Bcl-2 (sc-509) and Mcl-1 (sc-819) from Santa Cruz Biotechnology, Heidelberg, Germany; Tubulin and actin from Sigma, Saint Louis, USA.

### In Vivo Mouse Studies

Immunodeficient mice (Rag2−/−/II2rg−/−) were implanted subcutaneously in the dorsal flank region with CEM (5×107 cells). After the tumors reached to approximate 35 mm3, tumor size matched mice were treated with ABT-263 (50 mg/kg, once in each third or fourth day) in the presence or absence of 50 mg/kg wogonin. ABT-263 was formulated according to the protocol previously described 9 and administered p.o. Wogonin dissolved in DMSO was diluted in sunflower oil and administered i.p. For combination studies, ABT-263 was given 2 h before wogonin. The control group was treated in an analogous manner with the vehicle. The tumor size was measured with a micrometer caliper two to three times weekly and the tumor volume (V) was calculated by the formula V=(width2×length)/2. All protocols using and maintaining animals were approved by the German Animal Protection Authority (Office Regierungsprasidium Karlsruhe) in Karlsruhe.

### EXAMPLE 2

### Rocaglamide Sensitizes ABT-263-Induced Apoptosis in Leukemia Cells

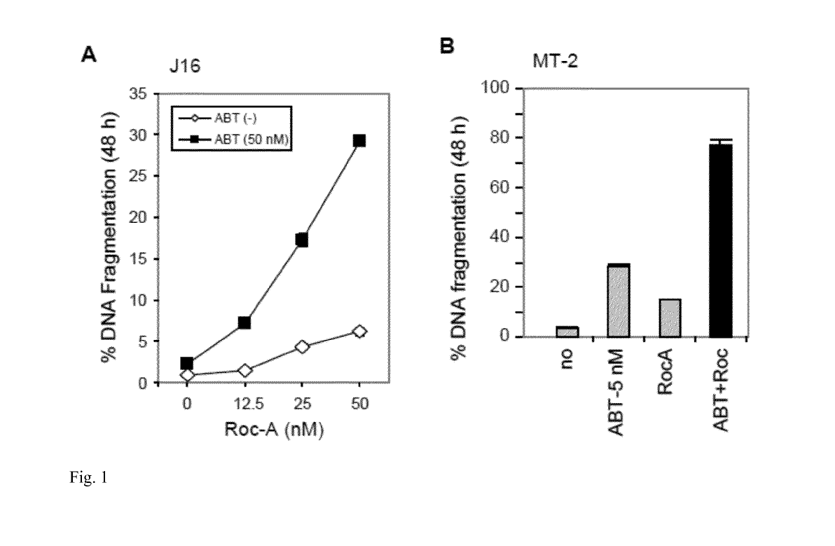

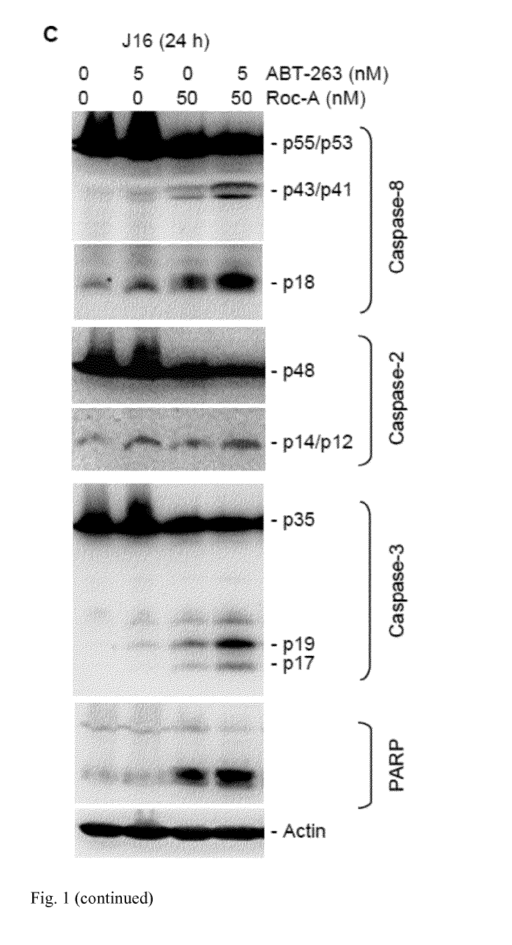

To investigate whether rocaglamide could enhance the toxicity of ABT-263 on leukemic cells, leukemic T cell line Jurkat and the HTLV-1-associated ATL cell line MT-2 were treated with indicated amounts of ABT-263 in the absence or presence of different concentration of rocaglamide for 48 h. Apoptotic cell death was determined by DNA fragmentation. As shown in FIGS. 1A and B, ABT-263-induced apoptosis was sensitized by rocaglamide in both leukemic cell lines in a dose-dependent manner. Significant enhancement of activities of caspase-8, caspase-3 and increase in PARP cleavage was seen after 24 h by combination treatment by Western blot analysis (FIG. 1C).

### EXAMPLE 3

### Wogonin Enhances ABT-263-Induced Apoptosis in Leukemia Cells

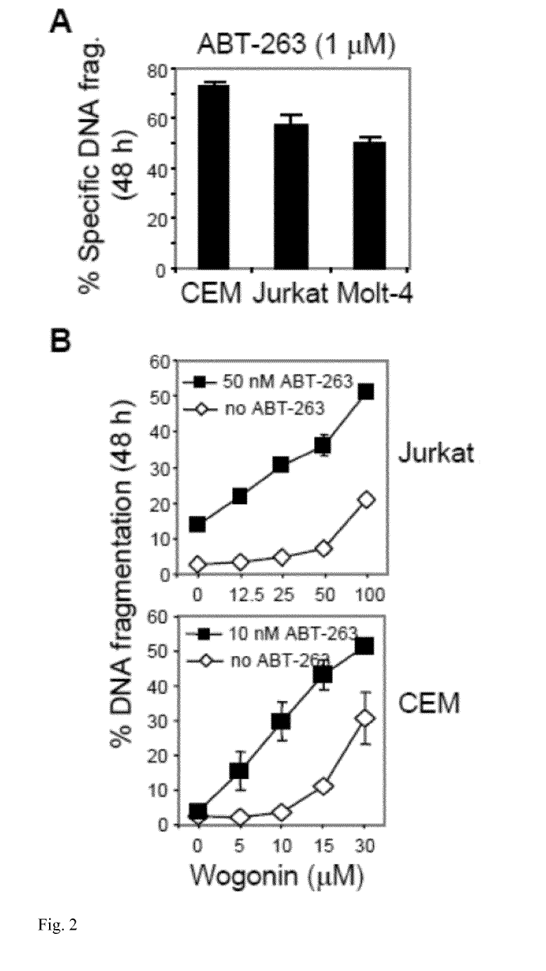

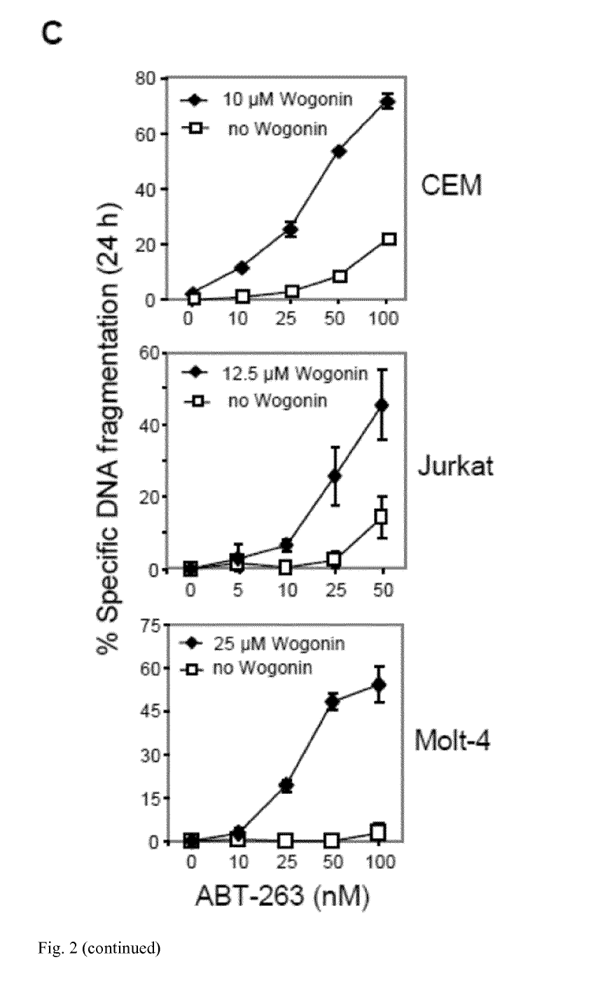

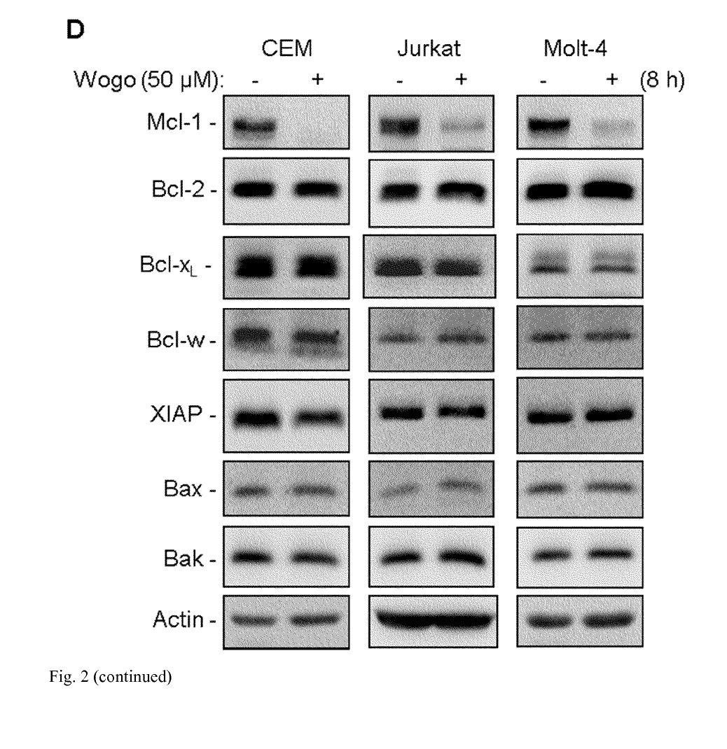

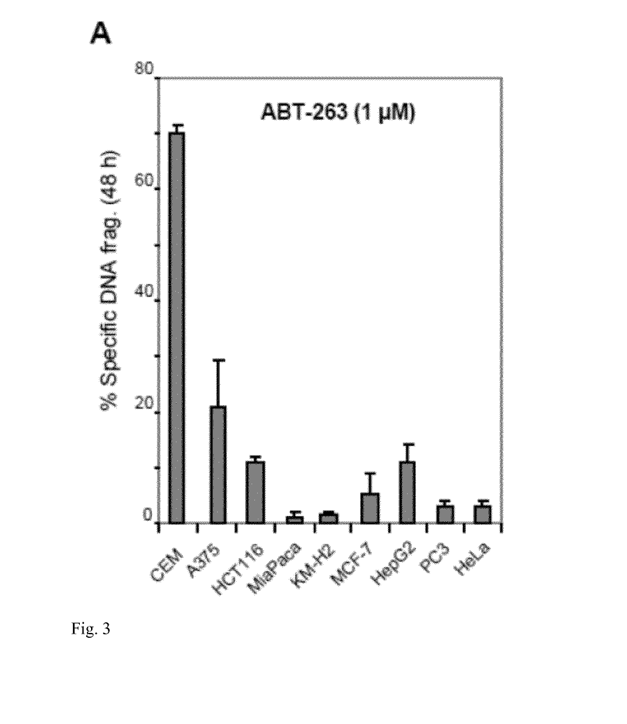

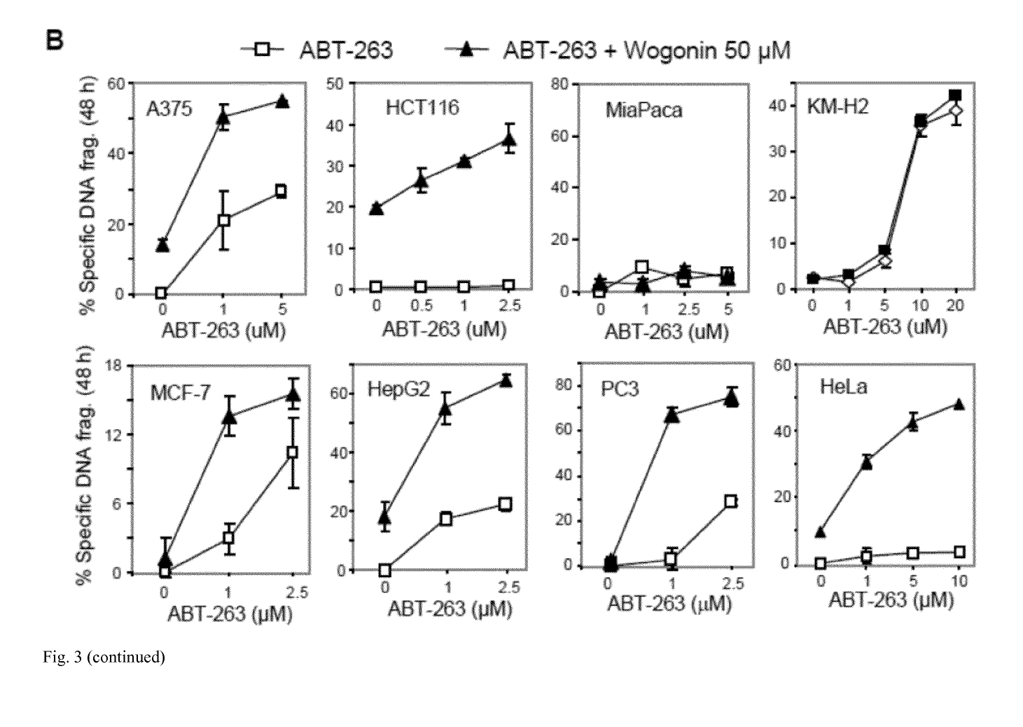

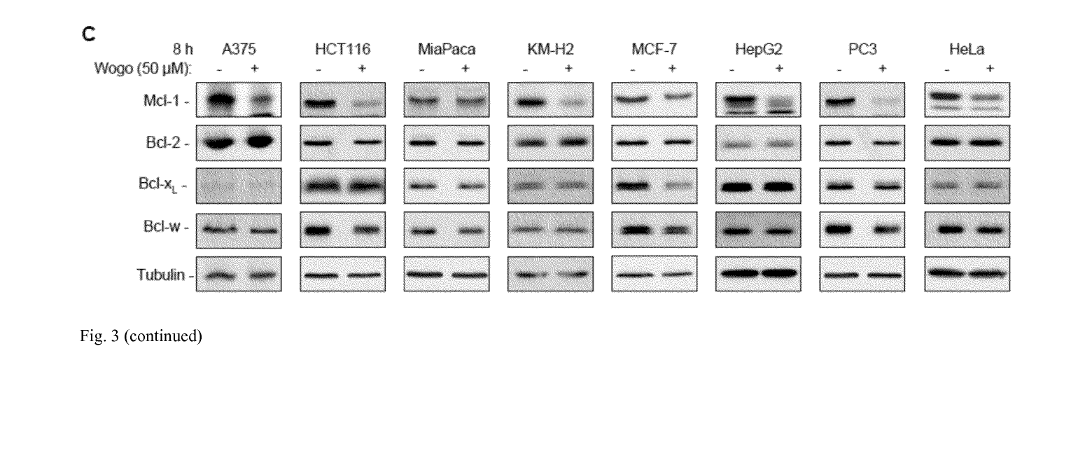

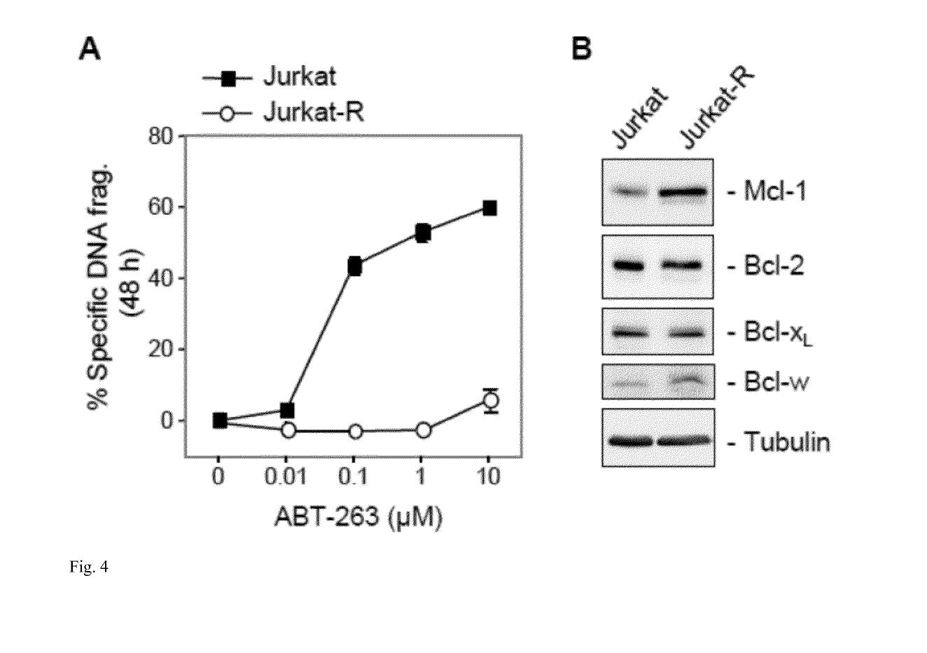

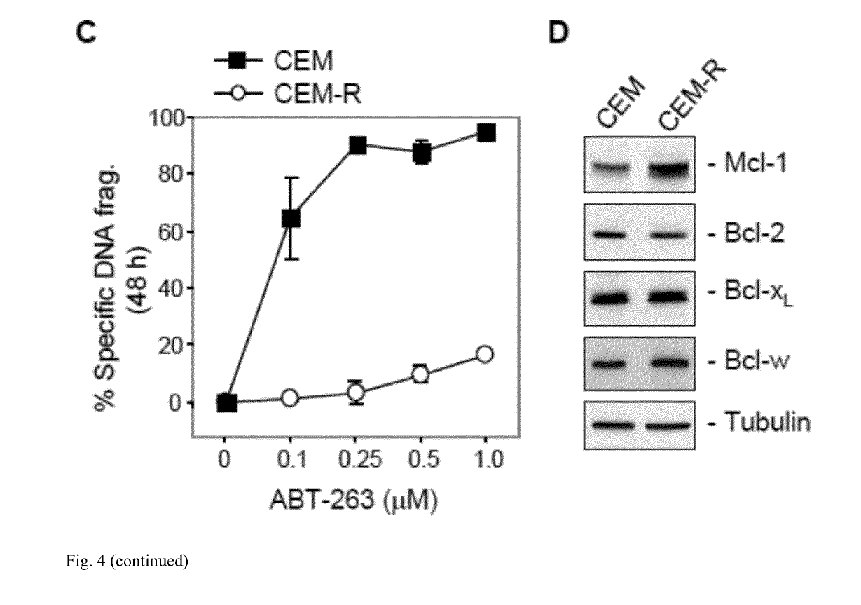

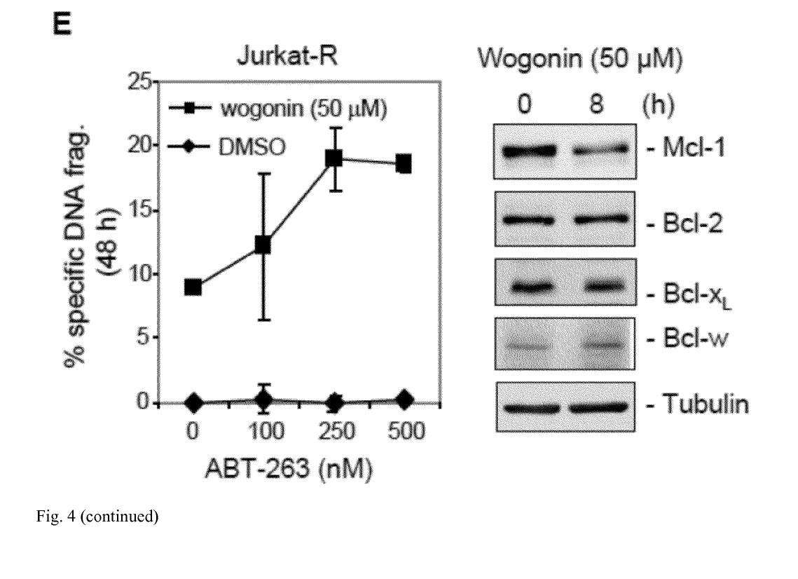

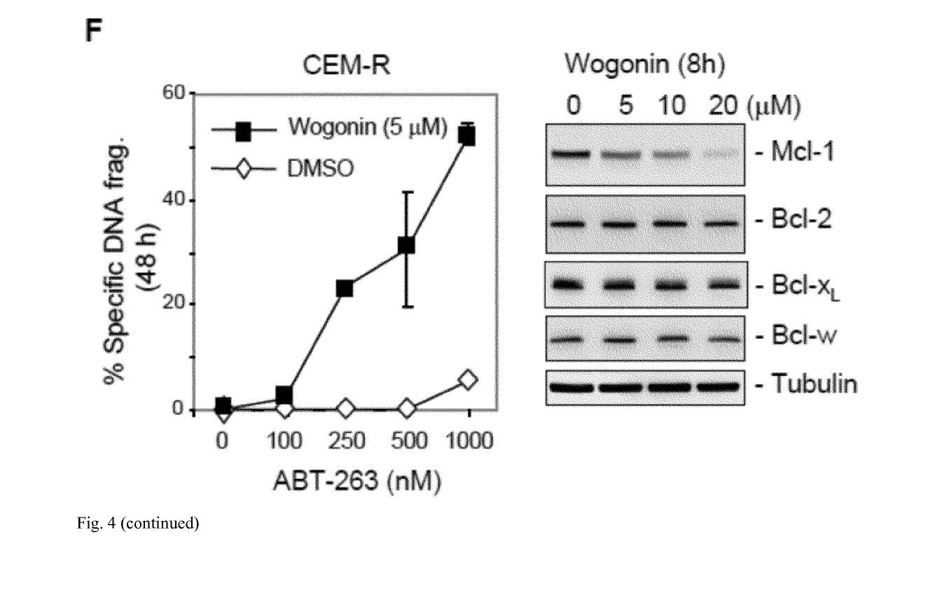

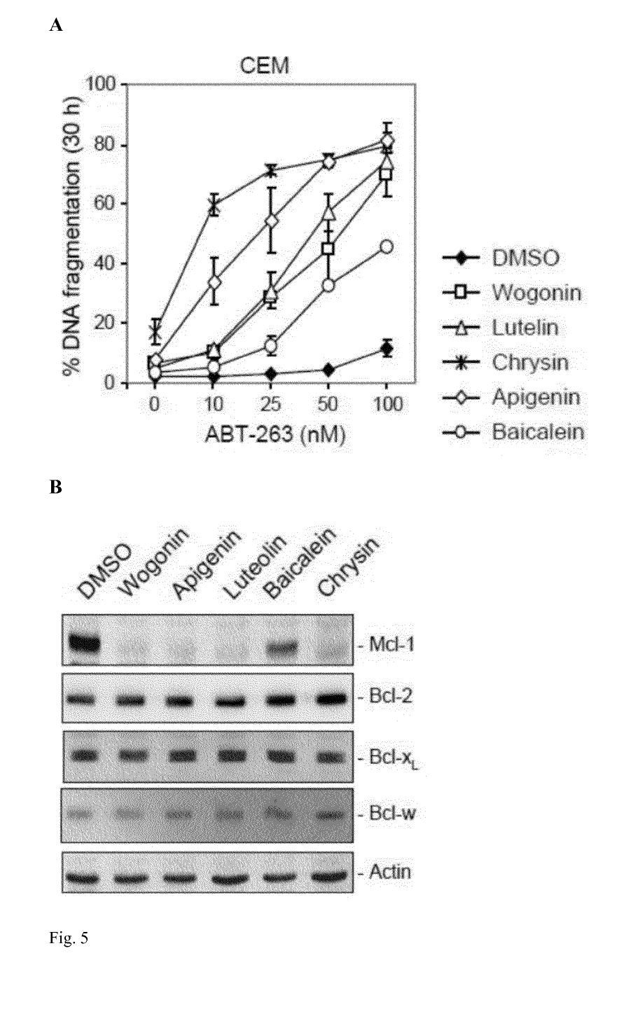

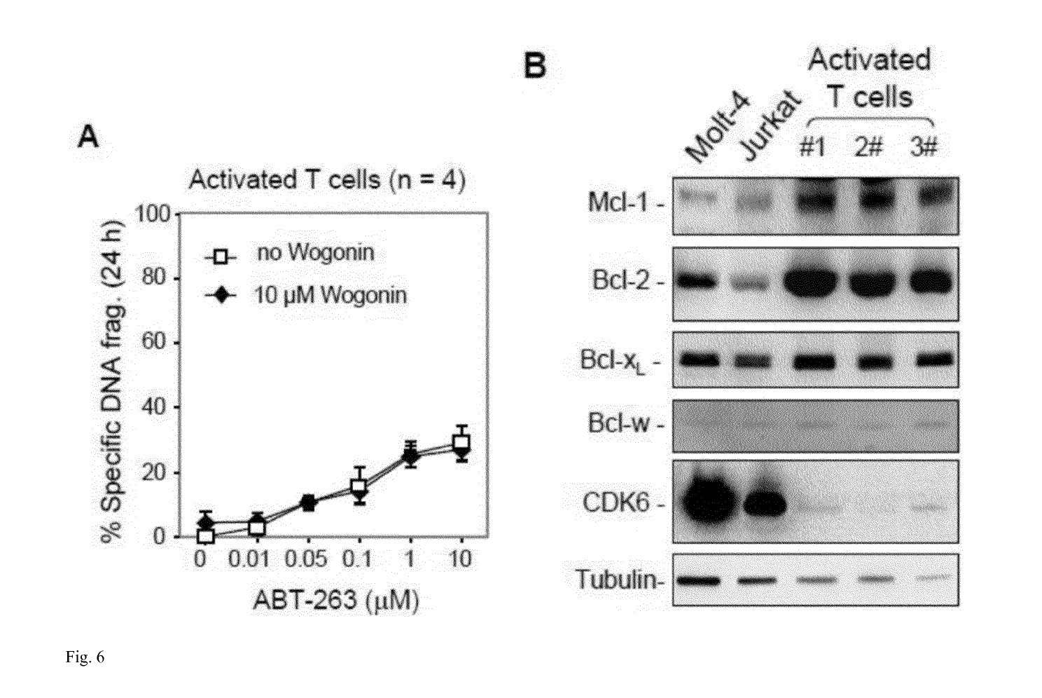

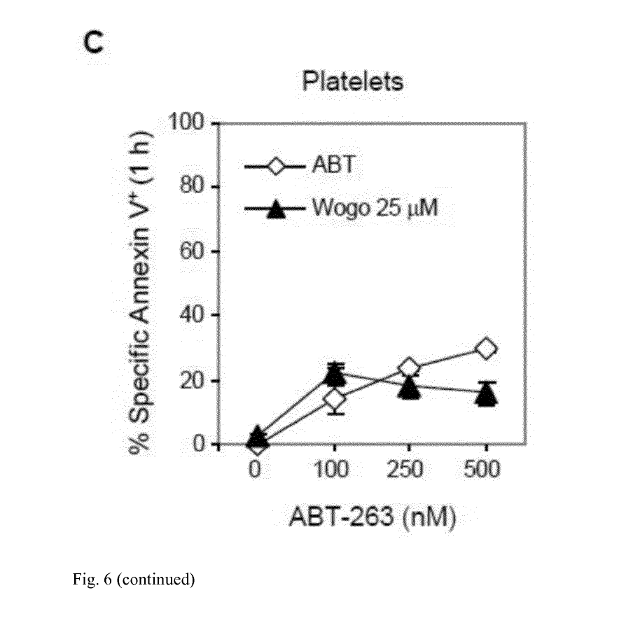

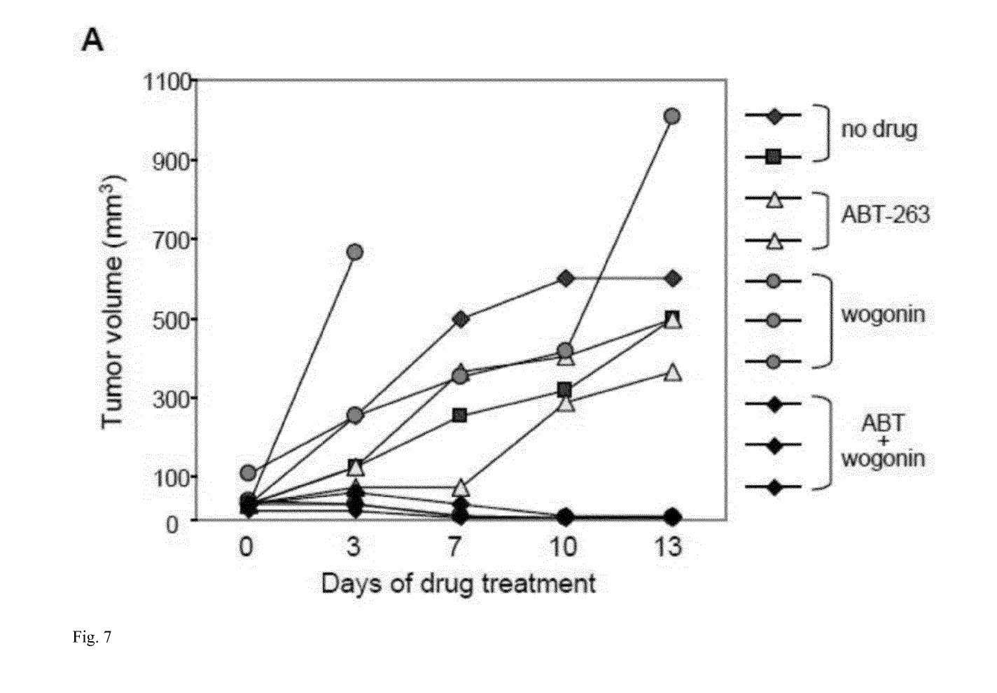

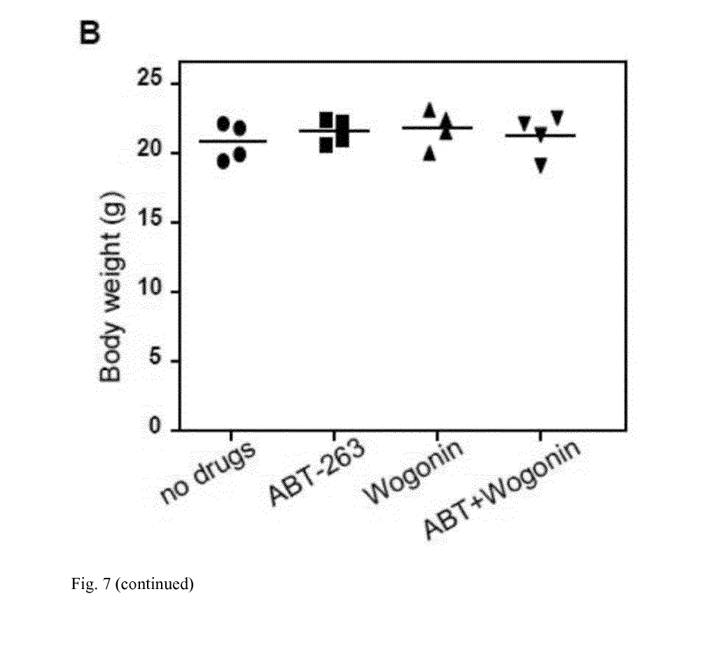

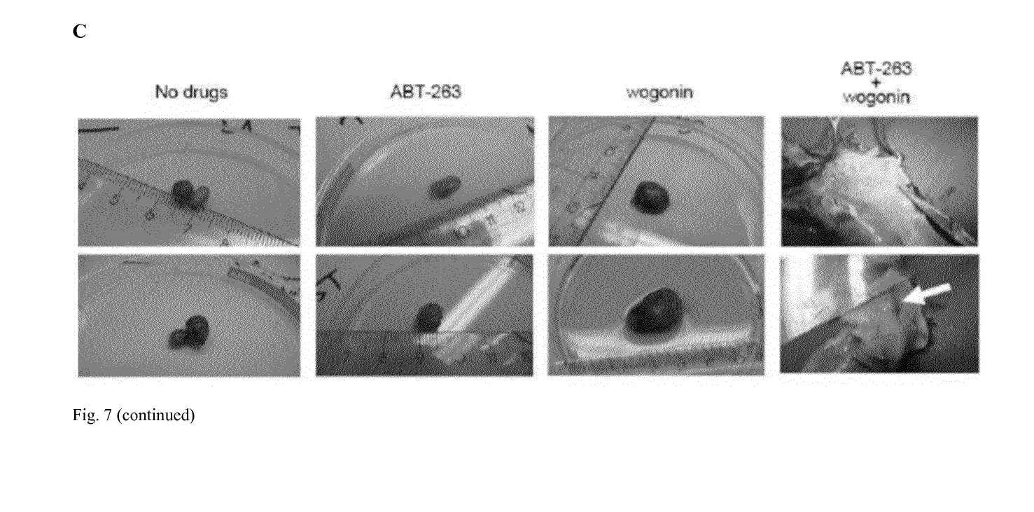

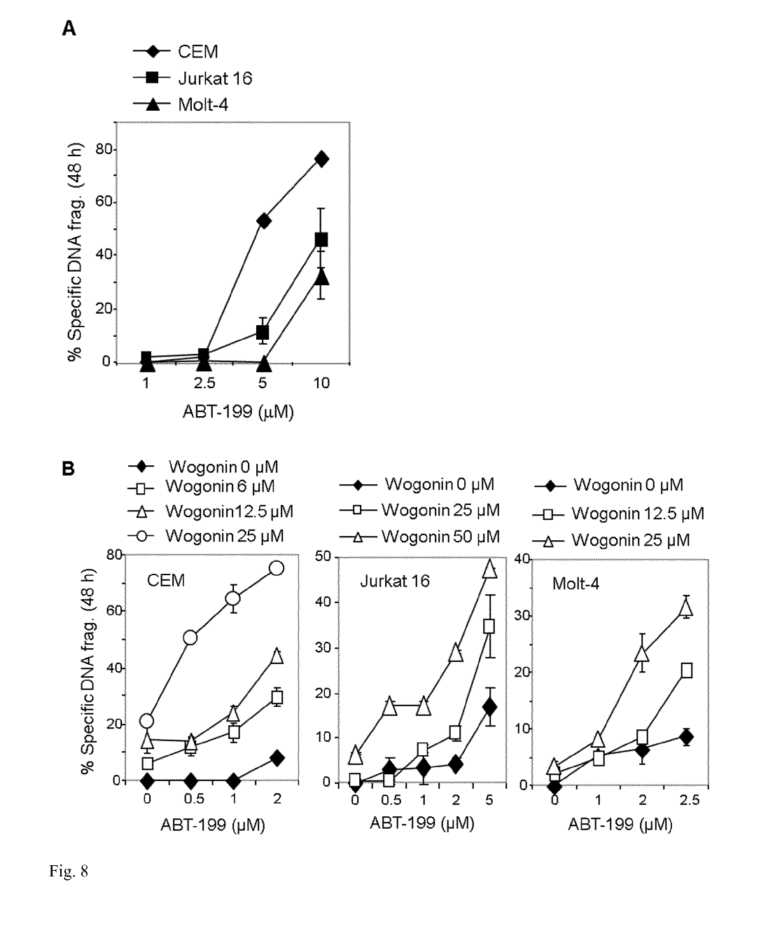

It was asked whether wogonin could enhance the toxicity of ABT-263 on leukemic cells. To answer this question, the three human leukemic T cell lines CEM, Jurkat and Molt-4 were used as a model system. Treatment with 1 μM of ABT-263 for 48 h resulted in apoptotic cell death in 50-70% of CEM, Jurkat and Molt-4 cells (FIG. 2A).

To investigate the effect of wogonin on ABT-263-induced cell death, CEM, Jurkat and Molt-4 with ABT-263 cells were treated in the absence or presence of different concentrations of wogonin. The experiments showed that wogonin, at the concentrations it alone induced only minimal cell death, enhanced the killing efficacy of ABT-263 in a dose-dependent manner in all three leukemic cell lines tested (FIGS. 2B and C). Wogonin significantly reduced the doses (approximately 10 times less) of ABT-263 required for inducing 50-70% cell death in leukemic cell lines tested (FIGS. 2B and C). Western blot analysis showed that wogonin-mediated down-regulation of Mcl-1 expression correlated with enhanced ABT-263 efficacy in all three cell lines tested (FIG. 2D).

### EXAMPLE 4

### Wogonin Potentiates the Efficacy of ABT-263 in De Novo Resistant Cancer Cells

The above experiments demonstrate that wogonin can enhance the killing efficacy of ABT-263 in leukemic cells. It was further asked whether wogonin could also enhance the toxicity of ABT-263 in other types of cancer cells and, in particular, ABT-263-insensitive cancer cells. To investigate this question, the effect of ABT-263 on CEM and other types of cancer cell lines including the human malignant melanoma cell line A375, the human colon carcinoma cell line HCT116, the human pancreatic cancer cell line MiaPaca, the human hepatocellular carcinoma cell line HepG2, the human Hodgkin lymphoma (HL) cell line KM-H2, the human breast cancer cell line MCF-7, the human prostatic cancer cell line PC3, and the human cervix adenocarcinoma cell line HeLa was compared. Many types of cancer cell lines display strong de novo resistance to ABT-263 compared to leukemic cell lines (FIG. 3A). Treatment with 1 μM ABT-263 for 48 h, which induced cell death in more than 50% of the leukemic cells (FIG. 2A), resulted in only 10-20% cell death in A375, HCT116 and HepG3 cells and less than 5% cell death in KM-H2, MiaPaca, Colo-357, MCF-7, PC3 and HeLa cells (FIG. 3A).

It was then asked whether wogonin could sensitize ABT-263-induced cell death in the de novo resistant cancer cells. For this, all tumor cell lines were treated with either ABT-263 alone or in combination with 50 μM wogonin. The experiments showed that except for MiaPaca and KM-H2, combination treatment resulted in a dose-dependent increase in ABT-263-mediated apoptotic cell death (FIG. 3B). Western blot analysis showed that except in MiaPaca, wogonin down-regulated Mcl-1 expression in all cell lines tested (FIG. 3C). The data demonstrate that wogonin can potentiate the ABT-263 toxicity in many types of tumor cells.

### EXAMPLE 4

### Wogonin Overcomes Acquired ABT-263 Resistance in Tumor Cells

Recently, it was described that sensitive lymphoma cell lines can become resistant to ABT-737 during long-term exposure by elevation of Mcl-1 expression. Indeed, continuous treatment of Jurkat and CEM cells with increasing concentrations of ABT-263 for two to three months rendered these cells resistant to ABT-263 (FIGS. 4A-D). The acquired resistance was shown to correlate with elevated Mcl-1 expression (FIGS. 4B and D). Thus, these experiments confirmed that ABT-263-sensitive cancer cells can develop acquired resistance during therapy via selective up-regulation of Mcl-1.

The development of resistance to chemotherapeutic drugs is a major challenge for cancer treatment. Therefore, it was asked whether wogonin could re-sensitize cells which have acquired resistance to ABT-263. To answer this question, the resistant Jurkat and CEM cells were treated with ABT-263 in the absence or presence of wogonin. The experiments showed that wogonin significantly inhibited Mcl-1 expression in the resistant cell lines. Consequently, resistant Jurkat and CEM cells were re-sensitized to ABT-263 treatment (FIGS. 4E and F). These experiments demonstrated that wogonin can suppress Mcl-1 expression in cell lines displaying acquired resistance and re-sensitize them to ABT-263-induced apoptosis.

### EXAMPLE 5

### Wogonin-Related Flavones Promote ABT-263-Induced Apoptosis

To test if wogonin-related natural flavones such as apigenin, chrysin and luteolin also down-regulate Mcl-1 expression, CEM cells were treated with ABT-263 either in the absence or presence of different wogonin-related flavones. Apigenin, chrysin, luteolin and the wogonin derivative baicalein were shown to enhance ABT-263-induced apoptosis in CEM cells in a dose-dependent manner (FIG. 5A). Down-regulation of Mcl-1 protein expression by these flavones correlated with the observed sensitization of ABT-263-induced apoptosis in CEM cells (FIG. 5B).

### EXAMPLE 6

### Selectivity of Wogonin and ABT-263 in Normal Lymphocytes and Platelets

To investigate the tumor selectivity of the combination treatment, proliferating normal blood T cells isolated from four healthy donors were examined. Proliferating normal T cells exhibited a higher resistance to ABT-263 compared to leukemic cells (FIG. 6A and FIG. 2A). This feature is explained, at least in part, by the fact that normal proliferating T cells express Bcl-2 and Mcl-1 at much higher levels than leukemic T cells (FIG. 6B). Importantly, wogonin did not enhance the toxicity of ABT-263 to normal proliferating peripheral blood T cells (FIG. 6A). Consistent with the study of Vogler et al. (2011, as above), at the concentration between 100 and 500 nM ABT-263 killed approximate 15-20% platelets (FIG. 6C). However, wogonin did not enhance the toxicity of ABT-263 to platelets (FIG. 6C).

### EXAMPLE 7

### Wogonin Enhances the Efficacy of ABT-263 in Xenografted Human Leukemic Cells in Vivo

To investigate the above observation in vivo, the effect of wogonin on ABT-263-mediated anti-cancer activity in Rag2^{-/-}/II2rg^{-/-} immunodeficient mice xenografted with the human CEM leukemic cells was evaluated. After the tumors reached approximate 35 mm³, tumor size matched mice were treated without or with ABT-263 in the presence or absence of wogonin. ABT-263 was previously shown to induce complete tumor response rate in the human ALL RS4;11 mouse model at 100 mg/kg/day. Therefore, the dose of ABT-263 was reduced to 50 mg/kg/day for consecutive two weeks. The experiment showed that combination of ABT-263 with wogonin induced rapid and complete tumor responses. In contrast, treatment with ABT-263 or wogonin alone showed no significant inhibition of tumor growth (FIG. 7A). ABT-263 plus wogonin was well tolerated with no body weight loss (FIG. 7B). This data demonstrate that wogonin can potentiate the anticancer activity of ABT-263 in vivo.

### EXAMPLE 4

### Wogonin Sensitizes Tumor Cells to ABT-199

CEM, Jurkat 16 and Molt-4 tumor cells were treated with ABT-199 in the absence or presence of wogonin (FIG. 8). ABT-199 alone induced 50% apoptotic cell death in leukemic cell lines at 5 to 10 mM concentration (FIG. 8A). Wogonin sensitized cell lines to ABT-199-mediated apoptosis and made possible an approx. 5fold reduction of the ABT-199 dose (FIG. 8B). Thus, the experiments showed that wogonin significantly increased sensitivity of the cell lines to ABT-199 treatment.

1-15. (canceled)

16. A method of treating cancer in a subject afflicted with cancer, comprising administering a combined preparation for simultaneous, separate or sequential use comprising
a) at least one flavagline or a pharmaceutically acceptable salt thereof; and/or at least one 5-hydroxy-flavone or a pharmaceutically acceptable salt thereof; and
b) at least one agent activating the intrinsic pathway of apoptosis.

17. The method according to claim 16, wherein the agent activating the intrinsic pathway of apoptosis is an inhibitor of at least one anti-apoptotic member of the Bcl-2 family of proteins.

18. The method according to claim 16, wherein the agent activating the intrinsic pathway of apoptosis is a BH3 mimetic small molecule inhibitor.

19. The method according to claim 16, wherein the flavagline is a compound of the formula (I) 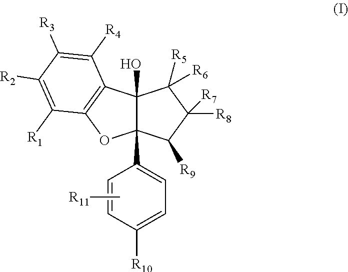
R₁ is selected from —H, halogen and alkyl;
R₂ is selected from alkoxy, halogen, and alkyl;
R₃ is selected from —H, halogen and alkyl;
or R₂ and R₃ together form a —O(CH₂)ₙO— unit, with n=1 or 2;
R₄ is selected from alkoxy, halogen, and alkyl;
R₅ is selected from hydroxyl, acyloxy, amino, monoalkylamino, dialkylamino and —NR₁₂—CHR₁₃—COOR_{14,} with
R₁₂ being selected from —H and alkyl,
R₁₃ being selected from phenyl and benzyl, which both may carry a substituent from the group hydroxyl, indolyl and imidazolylmethyl, and alkyl which may be substituted by a group selected from —OH, —SH, alkoxy, thioalkoxy, amino, monoalkylamino, dialkylamino, carboxy, carboxyalkyl, carboxamide and guanidino groups;
or R₁₂ and R₁₃ together form a —(CH₂)₃— or —(CH₂)₄— group;
R₁₄ being selected from alkyl and benzyl; in which case R₆ is hydrogen,
R₆ is selected from —H, halogen and alkyl;
or R₅ and R₆ together form an oxo or hydroxyimino group;
R₇ is —H;
R₈ is selected from —CONR₁₆R₁₂, —H, and —COOR₁₅ wherein
R₁₅ and R16 are independently selected from methyl and —H, and
R₁₇ is selected from methyl, —H, 4-hydroxybutyl and 2-tetrahydrofuryl;
R₉ is selected from phenyl which is optionally substituted, and hetaryl which is optionally substituted;
R₁₀ is selected from alkoxy, —H, halogen, and alkyl, and
R₁₁ is selected from —H, hydroxyl, halogen, alkoxy and alkyl;
or R₁₀ and R₁₁ are in ortho-position to each other and together form a —O(CH₂)ₙO— unit, with n=1 or 2.

20. The method according to claim 16, wherein the flavagline is (1R,2R,3S,3aR,8bS)-1,8 b-dihydroxy-6,8-dimethoxy-3a-(4-methoxyphenyl)-N,N-dimethyl-3-phenyl-2,3-dihydro-1H-cyclopenta[b][1]benzofuran-2-carboxamide (Rocaglamide A) or a derivative thereof.

21. The method according to claim 16, wherein the 5-hydroxy-flavone is of the formula (II) 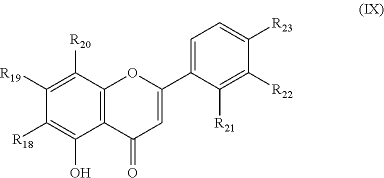
wherein
R₁₈ is —H, —OH, —CH₃, —CH₂OH, —OCH₃, phenyl, or hydroxyl-substituted phenyl,
R₁₉ is —OH, —H, —CH₃, —CH₂OH, —OCH₃, phenyl, or hydroxyl-substituted phenyl,
R20 is —OCH₃, —H, or a heterocyclic group (P) 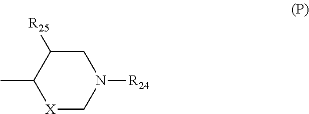
wherein X is —CH₂—, —O—, or —CH(OH)—, R₂₄ is —H, —CH_{3,} or —OCH_{3,} R₂₅ is —H or —OH,
R₂₁ is —H, —OH, —OCH₃, or —NH₂,
R₂₂ is —H, —OH, or —OCH₃, and
R₂₃ is —H or —OH, or —OCH₃;
preferably, is 5,7-dihydroxy-8-methoxy-2-phenyl-4H-chromen-4-one (Wogonin) or a derivative thereof.

22. The method according to claim 16, wherein the cancer is leukemia, lymphoma, preferably non-Hodgkin lymphoma, or small cell lung cancer.

23. The method according to claim 16, wherein the cancer is a cancer insensitive to an agent activating the intrinsic pathway of apoptosis.

24. The method according to claim 16, wherein the cancer is a cancer overproducing Mcl-1, in particular overproducing Mcl-1 but not an inhibitor of apoptosis acting downstream of the anti-apoptotic members of the Bcl-2 family of proteins.

25. The method according to claim 16, wherein said at least one agent activating the intrinsic pathway of apoptosis is administered at a dose avoiding severe side effects.

26. The method according to claim 16, wherein said at least one agent activating the intrinsic pathway of apoptosis is administered at a dose avoiding thrombocytopenia.

27. A combined preparation for simultaneous, separate or sequential use in the treatment of cancer comprising
a) at least one flavagline or a pharmaceutically acceptable salt thereof; and/or at least one 5-hydroxy-flavone or a pharmaceutically acceptable salt thereof; and
b) at least one agent activating the intrinsic pathway of apoptosis.

28. A medicament for the treatment of cancer which contains
a) least one flavagline or a pharmaceutically acceptable salt thereof; and/or at least one 5-hydroxy-flavone structure or a pharmaceutically acceptable salt thereof; and
b) at least one agent activating the intrinsic pathway of apoptosis,
and at least one pharmaceutically acceptable carrier.

29. A kit comprising a combined preparation according to claim 27 and a means for administering at least one of its components.

30. A kit comprising a medicament according to claim 28 and a means for administering at least one of its components.

31. A method of inhibiting a cancer cell, comprising
a) contacting said cancer cell with the combined preparation of claim 27, and
b) thereby inhibiting said cancer cell.

32. The method of claim 18, wherein the BH3 mimetic small molecule inhibitor is (R)-4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthio)butan-2-yl)amino)-3-((trifluoromethyl)sulfonyl)phenyl)sulfonyl)benzamide (ABT-263), 4-[4-[(4′-chloro[1,1′-biphenyl]-2-yl)methyl]-1-piperazinyl]-N-[[4-[[(1R)-3-(dimethylamino)-1-[(phenylthio)methyl]propyl]amino]-3-nitrophenyl]sulfonyl]-Benzamide (ABT-737), or 4-[4-[[2-(4-chlorophenyl)-4,4-dimethyl-1-cyclohexen-1-yl]methyl]-1-piperazinyl]-N-[[3-nitro-4-[[(tetrahydro-2H-pyran-4-yl)methyl]amino]phenyl]sulfonyl]-2-(1H-pyrrolo[2,3-b]pyridin-5-yloxy)-Benzamide (ABT-199).

33. The method of claim 23, wherein the cancer is a cancer insensitive to an agent activating the intrinsic pathway of apoptosis is a cancer insensitive to an inhibitor of Bcl-2, and/or Bcl-x_{L} and/or Bcl-w.
